(19) United States
(12) Patent Application Publication
(10) Pub. No.: US 2023/0405073 A1
(43) Pub. Date: Dec. 21, 2023

(54) Soft Chewable Compositions

(71) Applicant: The Procter & Gambie Comany, Cincinnati, OH (US)

(72) Inventors: Maria Delores Martinez Serna VILLAGRAN, Mason, OH (US); Rajesh K. Bund, Mason, OH (US); James Hepp, Cincinnati, OH (US); Erin Nicole Swigart, Morrow, OH (US); Valerie Naughton, Cincinnati, OH (US)

(21) Appl. No.: 18/447,348

(22) Filed: Aug. 10, 2023

Related U.S. Application Data
(63) Continuation of application No. 15/614,648, filed on Jun. 6, 2017.
(60) Provisional application No. 62/351,680, filed on Jun. 17, 2016.
(60) Provisional application No. 62/417,359, filed on Nov. 4, 2016.

(51) Int. Cl. A61K 36/68; A23L 33/22; A23G 4/06; A61K 9/68; A61K 9/50; C08L 5/06

(52) U.S. Cl. CPC A61K 36/68; A23L 33/22; C08L 5/06; A61K 9/0058; A61K 9/5036; A23G 4/068

(57) ABSTRACT

A soft chewable composition containing psyllium. The soft chewable composition can have from about 1% to about 55% psyllium and have a Hardness Parameter of greater than about 300 gf at a water activity of about 0.80 and less than about 10,000 gf at a water activity of about as measured by the Texture Profile Analysis Method.

### FIELD OF THE INVENTION

The present invention is directed towards a soft chewable composition containing psyllium, and methods of making and using such a composition.

### BACKGROUND OF THE INVENTION

Psyllium is a natural source of dietary fiber that has proven to have health benefits when consumed daily and following the recommended dose. Soluble viscous fiber, such as psyllium, can promote digestive health by relieving constipation and normalizing bowel movements and can help to curb hunger, maintain healthy blood sugar levels, and lower blood cholesterol. However, the average adult in the United States often ingests only about half of the recommended daily dose of fiber. One reason for low compliance is the inconvenience of current powder-form psyllium containing products, which must be mixed in a glass of water and consumed immediately before the beverage becomes thick. Many consumers are interested in consuming more psyllium due to its health benefits, but want a source of fiber in a convenient form, such as a soft chewable composition.

However, incorporating high levels of psyllium into a soft chewable product can be difficult due to the physical attributes of psyllium, particularly dispersibility, swelling, gelling, and viscosity. Although psyllium is capable of forming a weak gel in water, in a concentrated form, such as in a soft chewable composition, this gel can become hard and grainy during the shelf life of the product. In addition, psyllium has an excellent water absorption capacity, which can result in products without enough free water to provide lubricity and to soften the texture. Finally, loading psyllium into a soft chewable product at the daily dose recommended to deliver health benefits and at a dosage size acceptable to consumers can create an undesirable texture. As psyllium concentration is increased, the texture of the soft chewable composition becomes hard, grainy, and can result in toothpacking and sticking on oral surfaces when consumed, which further limits its use in consumer products.

Therefore, there is a need for a palatable, consumer acceptable, soft chewable composition that can provide high amounts of psyllium in a convenient form, as well as methods of making and using such a composition.

### SUMMARY OF THE INVENTION

A soft chewable composition comprising: from about 1% to about 55% psyllium; wherein the soft chewable composition has a Hardness Parameter of greater than about 300 gf at a water activity of about 0.80 and less than about 10,000 gf at a water activity of about 0.50 as measured by the Texture Profile Analysis Method.

A soft chewable composition comprising: (a) psyllium; (b) less than about 20% binding agent, wherein the binding agent is selected from the group consisting of pectin, gelatin, starch and combinations thereof; and (c) a processing aid; wherein the psyllium is substantially free of particles greater than about 250 μm; wherein the soft chewable composition comprises a final water activity from about 0.50 to about 0.80.

A method of making a soft chewable composition comprising: (a) preparing a syrup pre-mixture comprising a humectant component and a carbohydrate; (b) heating the syrup pre-mixture to form a cooked syrup pre-mixture; (c) adding a processing aid to the cooked syrup pre-mixture and mixing until the processing aid is melted; (d) adding psyllium to the cooked syrup pre-mixture and mixing to form a final mixture; (e) optionally heating the final mixture to a temperature required to obtain a desired solids content; (f) forming the final mixture into a soft chewable composition; and (g) optionally post-processing the soft chewable composition.

### BRIEF DESCRIPTION OF THE DRAWINGS

The patent or application file contains at least one photograph executed in color. Copies of this patent or patent application publication with color photograph(s) will be provided by the Office upon request and payment of the necessary fee.

While the specification concludes with claims particularly pointing out and distinctly claiming the subject matter of the present invention, it is believed that the invention can be more readily understood from the following description taken in connection with the accompanying drawings, in which:

FIGS. 1A-1H are photographs showing the effects of increasing psyllium concentration on the texture and appearance of soft chewable compositions;

FIGS. 2A-2E are photographs showing the effects of psyllium particle size on the texture and appearance of soft chewable compositions made in a starch mold;

FIGS. 3A-3C are photographs showing the effects of increasing psyllium concentration on the texture and appearance of low water soft chewable compositions; and

FIGS. 4A-4B are photographs showing the effects of psyllium particle size on the texture and appearance of low water soft chewable compositions.

### DETAILED DESCRIPTION OF THE INVENTION

Consumers are looking for convenient ways to incorporate more fiber into their diets. Soft chewable compositions, including but not limited to gummies and soft chews, are a fast and convenient supplement form that can include fiber. However, current fiber containing soft chewable products are made with inulin, which is a low viscosity syrup that does not form a viscous gel in water and does not provide the same health benefits as psyllium. It is challenging to incorporate psyllium into a soft chewable composition because it forms a viscous gel in water, which is difficult to handle during processing (e.g., pumping, filling into molds, etc.) and creates a grainy and hard texture at high concentrations.

It has been surprisingly found that the gelling properties of psyllium can be advantageously used along with particular levels and ratios of binding agents and processing aids to create a soft chewable composition with texture properties known to be acceptable to consumers. It has further been found that a soft chewable composition can be formulated with psyllium of a particular particle size distribution at a level that can deliver a daily dose of psyllium in a consumer acceptable dosage size, whilst still providing an acceptable texture and flavor.

The invention relates to a palatable, bite-sized soft chewable composition comprising psyllium. In one example, a soft chewable composition can include psyllium wherein the psyllium is substantially free of particles greater than about 250 μm, a humectant component, and a processing aid, wherein the soft chewable composition comprises an Aw from about 0.50 to about as packaged. In one example, the soft chewable composition can also include a binding agent selected from the group consisting of gelatin, starch, pectin, calcium salts, and combinations thereof. In one example, the soft chewable composition does not need a binding agent because the psyllium gel can act as a binder and can provide sufficient structure to create an acceptable texture.

As used herein, “adhesiveness” refers to a samples tendency to stick or adhere to a probe or surface and the force required to separate the sample from surface.

As used herein, “chewable” refers to a solid form, which can be taken by mouth and crushed into smaller pieces before swallowing

As used herein, “cohesiveness” refers to how well a composition withstands multiple compressions.

As used herein in the Examples, “DE” means “dextrose equivalent”, which refers to the percent of reducing sugars in a hydrolyzed starch, calculated as dextrose on a dry basis. Glucose (or corn) syrups are formed by reacting a starch with an acid and/or an enzyme. DE is a measurement of the degree of hydrolysis that starches undergo. Standard corn syrups generally have a DE of about 36 to 63. The higher the DE, the sweeter the component. However, higher DE also can contribute to a composition's greater tendency to crystallize, tendency to discolor, and tendency to be more hygroscopic, and can result in lower viscosity.

As used herein, “dietary fiber” or “fiber” refers to the fibrous or gummy component of food that is indigestible and non-metabolizable by humans. Fiber can include soluble fiber, which dissolves in water and insoluble fiber, which do not dissolve in water. Insoluble fiber can be metabolically inert and can provide bulking properties to food and/or prebiotic benefits.

As used herein, “dough” refers to a homogenous mixture that is a semi-solid.

As used herein “gumminess” refers to the force required to disintegrate a semi-solid food composition to a state ready for swallowing.

As used herein, “hardness” refers to the maximum force reached to complete the first compression of the sample.

As used herein a “humectant” refers to a substance having an affinity for water and which provides stabilizing action on the water content of a material. Humectants prevent loss of moisture from foods and prevent sugar from crystallizing. Humectants can also replace water in the formula while still keeping the desired plasticity for processing and the target texture.

As used herein, “psyllium” refers to ground psyllium or ispaghula husk. Psyllium is from the seeds of Plantago ovata or Plantago psyllium. In one example, the psyllium husk is from Plantago ovata.

As used herein, “room temperature” refers to a temperature of about 23 degrees Celsius (° C.).

As used herein, “springiness” refers to how well a composition physically springs back after it has been deformed during the first compression before the second compression. The spring-back is measured at the down-stroke of the second compression. This process emulates the sensory chewing experience. Thoroughly chewed foods generally do not have sufficiently remaining structural integrity to spring back (e.g. JELL-O®). The more a composition is destroyed, the less springiness it will exhibit.

As used herein, “swell volume” refers to the volume of gel mass formed when 0.5 g psyllium or psyllium containing products are mixed with water to a total volume of 100 mL. Swell volume provides a measure of the ability of the psyllium to absorb water.

As used herein, “water activity” (Aw) of a specimen refers to the ratio of the partial pressure of water vapor in equilibrium with that specimen at a particular temperature to the partial pressure of water vapor in equilibrium with pure water at that same temperature.

As used herein, the articles “a” and “an” are understood to mean one or more of the material that is claimed or described.

All weights, measurements and concentrations herein are measured at 23° C. and 50% relative humidity (RH), unless otherwise specified.

All percentages, parts and ratios as used herein are by weight of the total soft chewable composition, unless otherwise specified. All such weights as they pertain to listed ingredients are based on the active level and, therefore do not include solvents or by-products that may be included in commercially available materials, unless otherwise specified.

Consumer acceptable soft chewable products can have a range of texture attributes including hardness, gumminess, springiness, cohesiveness, and adhesiveness. For instance, soft chewable compositions with a Hardness Parameter of from about 1,000 to about 10,000 gram-force (gf) at the time of purchase can be acceptable to consumers. In some cases, the Hardness Parameter of a soft chewable composition can be below or above this range and still be consumer acceptable depending on the other texture attributes. For instance, a Hardness Parameter from about 400 gf to about 15,000 gf may still be consumer acceptable. For soft chewable compositions, the hardness of the product can change as a function of the final solids content, age of the composition, level of plasticizers in the formula, water activity, relative humidity, and/or the temperature of storage.

A consumer acceptable soft chewable composition can have a Hardness Parameter of greater than about 1,000 gf at a water activity of about 0.80 and less than about 6,000 gf at a water activity of about 0.50. A consumer acceptable low water soft chewable composition can have a Hardness Parameter of greater than about 6,000 gf at a water activity of about 0.80 and less than about 10,000 gf at a water activity of about 0.50.

While hardness is one of the biggest texture drivers of consumer acceptance for a soft chewable composition, it is not the only factor. The other texture attributes must also be balanced in order to create a soft chewable composition that consumers will find acceptable. For instance, consumers prefer soft chewable compositions that have high springiness because the texture is soft and the composition springs back after chewing. Gumminess can also be desirable because it can provide a good mouth melt and prevent toothpacking or sticking. A soft chewable composition with a high cohesiveness can also be preferred, as compositions with a low cohesiveness can be dry and crumble. In addition, a composition with strong cohesion will be more tolerant of manufacturing, packaging and delivery stresses, and thus will be presented to the consumers in its expected state. Finally, low adhesiveness can be desirable because as adhesiveness increases, compositions can stick together and to packaging and can stick to teeth and gums when consumed.

A consumer acceptable soft chewable composition can have a Cohesiveness Parameter of about 0.40 to about 0.90, a Springiness Parameter in the range of about 0.60 to about 0.90, a Gumminess Parameter of from about 1,000 to about 4,000 gf, and/or an Adhesiveness Parameter of about −1000 gf s to about 0 gf s at the time of purchase. Consumers may also find soft chewable products with a higher or lower gumminess to be acceptable, for instance having a Gumminess Parameter as low as 300 gf or as high as 6,000 gf. In addition, a consumer may also find a soft chewable product with an Adhesiveness Parameter of about −400 gf s to be acceptable, depending on the balance of other texture attributes, such as gumminess. A consumer may find a soft chewable composition with an Adhesiveness Parameter of about −50 gf s to be acceptable if the Gumminess Parameter is as high as about 3,000 gf.

Psyllium Level

Different formulas were tested to assess the impact of psyllium on the texture and appearance of a soft chewable composition. Examples 1-6 were made according to the procedure described hereafter. The examples were made using gelatin as the binding agent and psyllium having particle sizes distributed as follows: about 100% of the particles less than about 250 μm, about 92% of the particles less than about 212 μm, about 83% less than about 180 μm, about 61% less than about 150 μm, about 30% less than about 106 μm, and about 11% less than about 75 μm.

Examples 1-6 were made according to the following formulas.

| | Ex. 1 | Ex. 2 | Ex. 3 | Ex. 4 | Ex. 5 | Ex. 6 |
|---|---|---|---|---|---|---|
| | (wt %) | (wt %) | (wt %) | (wt %) | (wt %) | (wt %) |
| Water | 12.22 | 11.98 | 14.36 | 13.84 | 11.12 | 10.03 |
| Corn Syrup 42 | 34.50 | 34.66 | 32.59 | 32.73 | 28.17 | 25.30 |
| DE | | | | | | |
| Sucrose | 29.19 | 29.30 | 27.24 | 27.36 | 28.83 | 24.57 |
| Crisco ® | 5.14 | 5.14 | 4.19 | 4.24 | 4.05 | 3.91 |
| Shortening¹ | | | | | | |
| Knox ® Gelatin | 3.74 | 3.73 | 2.56 | 2.59 | 1.31 | 0.98 |
| Citric Acid | 1.19 | 1.19 | 1.05 | 1.06 | 0.81 | 0.78 |
| Psyllium | 13.80 | 13.78 | 17.80 | 17.96 | 25.16 | 33.21 |
| Flavoring Agent | 0.11 | 0.11 | 0.11 | 0.11 | 0.50 | 1.17 |
| Coloring Agent | 0.11 | 0.11 | 0.10 | 0.11 | 0.05 | 0.05 |
| Total | 100.00 | 100.00 | 100.00 | 100.00 | 100.00 | 100.00 |
| kcal/100 g | 348 | 349 | 337 | 339 | 352 | 359 |
| (psyllium: | | | | | | |
| 4 kcal/g) | | | | | | |
| % Solid content | 80.95 | 81.31 | 79.27 | 79.55 | 83.58 | 85.10 |
| (Theoretical) | | | | | | |
| % Moisture | 19.05 | 18.69 | 20.73 | 20.45 | 16.42 | 14.90 |
| content | | | | | | |
| (Theoretical) | | | | | | |

¹Crisco ® Baking Sticks, Lot # 531342004 08:19 C

The examples were held for 4 days at room temperature and about 60% RH before ejecting from the molds. The examples were stored in covered glass jars until texture parameters were measured. Texture Parameters were measured according to the methods described hereafter.

| | Ex. 1 | Ex. 2 | Ex. 3 | Ex. 4 | Ex. 5 | Ex. 6 |
|---|---|---|---|---|---|---|
| Mold Type: | | | | | | |
| Polymer | | | | | | |
| Hardness | 3158 | | 4067 | | 5885 | 21396 |
| Parameter (gf) | | | | | | |
| Adhesiveness | −15.81 | | −1.49 | | −3.50 | −98.39 |
| Parameter (gf s) | | | | | | |
| Springiness | 0.85 | | 0.84 | | 0.56 | 0.74 |
| Parameter | | | | | | |
| Cohesiveness | 0.71 | | 0.68 | | 0.53 | 0.51 |
| Parameter | | | | | | |
| Gumminess | 2232 | | 2776 | | 3130 | 10888 |
| Parameter (gf) | | | | | | |
| Water Activity | 0.74 | | 0.81 | | 0.69 | 0.69 |
| (Aw) | | | | | | |
| Mold Type: | | | | | | |
| Starch | | | | | | |
| Hardness | | 5925 | | 7900 | 11618 | 29358 |
| Parameter (gf) | | | | | | |
| Adhesiveness | | −74.02 | | −8.97 | 0.00 | −1.55 |
| Parameter (gf s) | | | | | | |
| Springiness | | 0.82 | | 0.79 | 0.67 | 0.69 |
| Parameter | | | | | | |
| Cohesiveness | | 0.64 | | 0.63 | 0.52 | 0.51 |
| Parameter | | | | | | |
| Gumminess | | 3800 | | 4973 | 6040 | 15072 |
| Parameter (gf) | | | | | | |
| Water Activity | | 0.65 | | 0.66 | 0.64 | 0.60 |
| (Aw) | | | | | | |

It was surprisingly found that psyllium can be used as an effective binder to create a soft chewable composition. As the psyllium level in the formula increased, the level of gelatin binding agent and other plasticizers (e.g., shortening, sugar, corn syrup, and water) could be decreased.

It was further found that the type of molding used during processing can affect the final texture of the soft chewable composition. Soft chewable compositions created using a starch mold had a higher Hardness and Gumminess Parameter and a lower Aw as compared to soft chewable compositions created in a polymer mold. However, the Springiness and Cohesiveness Parameters did not change significantly between the two types of molds. It is believed that the increased hardness and gumminess in starch molds can be due in part to the gradient in moisture content from the center of the soft chewable composition to the surface of the soft chewable composition because starch can absorb the external excess moisture content during curing. Soft chewable compositions molded in a polymer mold do not show a gradient in moisture content, and therefore, the hardness and gumminess can be lower.

As the psyllium level increased, the hardness and gumminess of the soft chewable composition increased. Example 1, which had about 13% psyllium, had the lowest Hardness and Gumminess Parameters of about 3,158 gf and about 2,232 gf respectively, but still fell within the texture ranges known to be acceptable by consumers. Example 3, which had about 18% psyllium, had a higher Hardness and Gumminess Parameter as compared to Example 1, but the values still fell within the texture ranges known to be acceptable by consumers. At 25% psyllium, Example 5 had a Hardness Parameter of about 5,885 gf and a Gumminess Parameter of about 3,130 gf, which still fell within the levels known to be acceptable by consumers. However, at 34% psyllium the texture of the soft chewable composition was negatively affected. Example 6, which had the highest level of psyllium, had a Hardness Parameter of over 21,000 gf and a Gumminess Parameter of over 10,000 gf, which fell above the levels known to be acceptable by consumers.

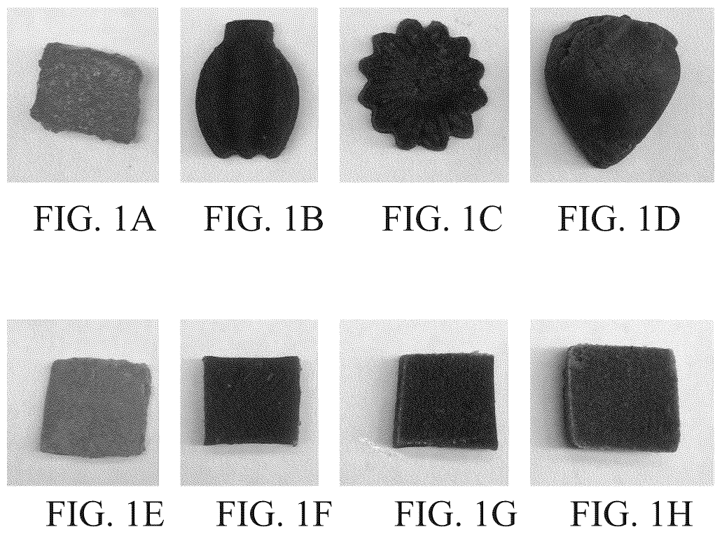

FIGS. 1A-1D show Examples 3, 5, 6 and 12, respectively, to demonstrate the effect of increasing psyllium concentration on the appearance and texture of soft chewable compositions made in a polymer mold. FIGS. 1E-1H show Examples 4, 5, 6 and 12, respectively, to demonstrate the effect of increasing psyllium concentration on the appearance and texture of soft chewable compositions made in a starch mold. Example 12 was made without psyllium and is described hereafter. It was found that as the psyllium level increased, the soft chewable composition had a harder, grainier appearance and the color became darker. It is thought that the change in color may be due to the brown color of psyllium and/or non-enzymatic browning as a result of the preparation of the psyllium and/or the cooking of the soft chewable composition.

It was found that an acceptable hardness could be achieved in some soft chewable composition formulations by using liquid fructose comprising about 70 to about 95% solids, preferably about 80%. It is believed that liquid fructose comprising solids within this range can soften the texture of the soft chewable composition and allow for increased levels of psyllium, for instance levels of psyllium of about 45%. Liquid fructose has a lower viscosity than corn syrup at the same percent of solids. It is believed that the substitution of liquid fructose for corn syrup can allow for the addition of higher levels of psyllium while achieving an acceptable hardness.

Psyllium Particle Size

Different formulas were tested to assess the impact of initial psyllium particle size distribution on the texture and appearance of a soft chewable composition. Examples 7-10 were made according to the procedure described hereafter. The examples were made using gelatin as the binding agent and 17% psyllium of varying initial particle size.

The psyllium particle sizes tested are described in the table below.

| Psyllium | Material | | | | | | | | | | | |
|---|---|---|---|---|---|---|---|---|---|---|---|---|
| Ingredients | Description | Particle Distribution | | | | | | | | | | |
| Screen Retention Size | | On | On | On | On | On | On | On | On | On | On | On |
| (Mesh) | | 18 | 20 | 25 | 30 | 40 | 60 | 70 | 80 | 100 | 140 | 200 |
| Micron Size (μm) | | >1000 | >850 | >710 | >600 | >425 | >250 | >212 | >180 | >150 | >106 | >75 |
| ST | Smooth | 0 | 0 | 0 | 0 | 0 | 0 | 0 | 0 | 1.6 | 37.1 | 26.2 |
| | Texture | | | | | | | | | | | |
| XT | Modified | 0 | 0 | 0 | 0 | 0 | 0 | 8.0 | 9.0 | 22.0 | 31.0 | 19.0 |
| | Smooth | | | | | | | | | | | |
| | Texture | | | | | | | | | | | |
| OT | Original | 0 | 0 | 0.1 | 0.2 | 8.7 | 40.3 | — | — | — | — | — |
| | Coarse | | | | | | | | | | | |
| | Texture | | | | | | | | | | | |
| Husk | Natural | 1.7 | 4.3 | 17.4 | 11.5 | 25.7 | 25.2 | — | — | — | — | — |
| | Texture | | | | | | | | | | | |

Psyllium Husk is the raw material which has the largest particle size. Psyllium Husk includes particle sizes distributed as follows: about 84% of the particles are less than about 1000 μm and greater than about 250 μm. Psyllium OT is psyllium that has been ground to a point where particle sizes are distributed as follows: about 49.2% of the particles are equal or less than about 710 μm and greater than about 250 μm. Psyllium XT was ground to a point where particle sizes are distributed as follows: about 100% of the particles are less than about 250 μm, about 92% of the particles are less than about 212 μm, about 83% are less than about 180 μm, about 61% are less than about 150 μm, about 30% are less than about 106 μm, and about 11% are less than about 75 μm. Psyllium ST was ground to the smallest particle size, with particle sizes distributed as follows: about 100% of the particles are less than about 180 μm, about 98.4% are less than about 150 μm, about 61.3% are less than about 106 μm, and about 35.1% are less than about 75 μm. Psyllium XT was coarser than ST, but significantly finer than OT and Husk. Particle size refers to unagglomerated psyllium particle size. Particle sizes and particle size distributions can be measured according to the Particle Size Method described hereafter.

Examples 7-10 were made according to the following formulas.

| | Ex. 7 | Ex. 8 | Ex. 9 | Ex. 10 |
|---|---|---|---|---|
| | Psyllium Type | | | |
| | Husk | OT | XT | ST |
| | (wt %) | (wt %) | (wt %) | (wt %) |
| Water | 9.73 | 10.15 | 9.82 | 10.61 |
| Corn Syrup 42 DE | 31.43 | 31.28 | 31.39 | 31.19 |
| Sucrose | 32.90 | 32.77 | 32.90 | 32.57 |
| Crisco ® Shortening¹ | 4.10 | 4.04 | 4.05 | 4.00 |
| Knox ® Gelatin | 2.54 | 2.52 | 2.54 | 2.51 |
| Citric Acid | 0.81 | 0.81 | 0.81 | 0.80 |
| Psyllium | 17.23 | 17.17 | 17.22 | 17.06 |
| Flavoring Agent | 1.21 | 1.21 | 1.22 | 1.21 |
| Coloring Agent | 0.05 | 0.05 | 0.05 | 0.05 |
| Total | 100.00 | 100.00 | 100.00 | 100.00 |
| kcal/100 g (psyllium: | 356.00 | 354.00 | 355.00 | 352.00 |
| 4 kcal/g) | | | | |
| % Solid Content | 84.20 | 83.80 | 84.10 | 83.30 |
| (Theoretical) | | | | |
| % Moisture Content | 15.80 | 16.20 | 15.90 | 16.70 |
| (Theoretical) | | | | |

¹Crisco ® Baking Sticks, Lot # 531342004 08:19 C.

The examples were held for 4 days at room temperature and about 60% RH before ejecting from the molds. The examples were stored in covered glass jars until texture parameters were measured. Texture Parameters were measured according to the methods described hereafter.

| | Ex. 7 | Ex. 8 | Ex. 9 | Ex. 10 |
|---|---|---|---|---|
| Mold Type: Polymer | | | | |
| Hardness Parameter (gf) | 1412 | 1450 | 2201 | 4210 |
| Adhesiveness Parameter | −752.83 | −918.92 | −857.05 | −445.33 |
| (gf s) | | | | |
| Springiness Parameter | 0.79 | 0.92 | 0.78 | 0.72 |
| Cohesiveness Parameter | 0.43 | 0.48 | 0.47 | 0.58 |
| Gumminess Parameter | 610 | 698 | 1042 | 2458 |
| (gf) | | | | |
| Water Activity (Aw) | 0.70 | 0.72 | 0.70 | 0.72 |
| Mold Type: Starch | | | | |
| Hardness Parameter (gf) | 2635 | 2709 | 2713 | 5196 |
| Adhesiveness Parameter | −451.69 | −222.32 | −91.13 | −0.10 |
| (gf s) | | | | |
| Springiness Parameter | 0.60 | 0.80 | 0.53 | 0.72 |
| Cohesiveness Parameter | 0.40 | 0.44 | 0.42 | 0.63 |
| Gumminess Parameter | 1053 | 1180 | 1126 | 3257 |
| (gf) | | | | |
| Water Activity (Aw) | 0.65 | 0.65 | 0.67 | 0.64 |

It was surprisingly found that initial psyllium particle size can affect the texture of the soft chewable composition. Although the texture parameters fell within the ranges known to be acceptable to consumers, when psyllium having an initial particle size distribution of about 84% less than about 1000 μm and greater than about 250 μm and/or about 49.2% equal or less than about 710 μm and greater than about 250 μm was used, it created a gritty mouthfeel and appearance.

When the soft chewable compositions were molded in a polymer mold, it was found that the Hardness Parameter decreased as the psyllium particle size increased. Example 10, which had psyllium ST, had the highest Hardness and Gumminess Parameters as compared to the examples having larger psyllium particles. When the soft chewable composition was made with XT psyllium, as in Example 9, the Hardness and Gumminess Parameters decreased as compared to Example 10. Examples 7 and 8, which had Psyllium Husk and OT respectively, had the lowest Hardness and Gumminess Parameters and also had a gritty mouthfeel and visible particles.

When the soft chewable compositions were molded in a starch mold, it was found that the Hardness Parameter decreased as the psyllium particle size increased and the Gumminess Parameter remained high across all psyllium particle sizes. It was further found that adhesiveness increased as the psyllium particle size increased. Examples 7 and 8 had Hardness and Gumminess Parameters that fell within the ranges known to be acceptable to consumers, but had a gritty mouthfeel and visible particles. Examples 9 and 10, which were made with psyllium XT and ST respectively, had higher Hardness and Gumminess Parameter values, as compared to Examples 7 and 8. Examples 9 and 10 had Hardness and Gumminess Parameter values that fell within the ranges known to be consumer acceptable and did not have a significantly gritty mouthfeel or appearance.

Without being limited by theory, it is believed that the large psyllium particles may not be sufficiently hydrated when added to the formula, and therefore are unable to completely interact with the gelatin or other binders in the formula to form a network. It is believed that psyllium with the highest level of smaller particles, such as in psyllium ST, have a higher hydration rate, and therefore are able to form a stronger gel than coarser material like Psyllium OT and Husk.

Comparing the methods of molding, it was found that starch molding can reduce the adhesiveness, as well as increases the Gumminess and Hardness Parameters as compared to polymer molding.

It was found that when measured 4 days after the soft chewable composition was made, the Adhesiveness Parameter across all initial psyllium particle size distributions and molding methods ranged from −451.69 to −0.10 gf s. While it is desired that the Adhesiveness Parameter is near zero, the ranges still fall within the ranges known to be acceptable to consumers. In addition, the known acceptable Adhesiveness Parameters are based on products which have been processed with a sugar or oil coating and have been on a shelf for an unknown period of time, which could cause adhesiveness to decrease. It is believed that adhesiveness may not be a major driver of texture, as adhesiveness can be impacted by post-processing steps and the time point at which the samples were tested.

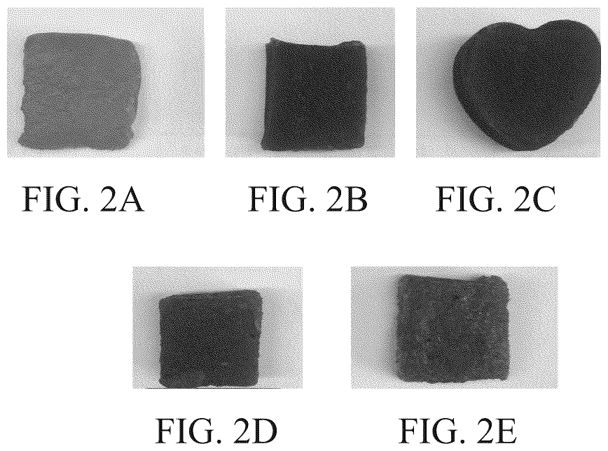

FIGS. 2A-2E show Examples 7, 8, 9, 10 and 12, respectively, to demonstrate the effect of psyllium particle size on the appearance and texture of soft chewable compositions made in a starch mold. Example 12 was made without psyllium and is described hereafter. It was found that as the initial psyllium particle size increased, the soft chewable composition had a more grainy appearance. For instance, Examples 9 and 10 have a smoother appearance than Examples 7 and 8, which have visible particles. It was also found that the color of the psyllium changed between Husk and the other ground psylliums. The ground psylliums are subjected to shear and temperature generated during milling, which may cause some level of non-enzymatic browning as shown in FIGS. 2A-2E.

Impact of Psyllium

Different formulas were tested to assess the impact of psyllium on the texture of a soft chewable composition. Example 9 was made as described above and Examples 11-13 were made according to the procedure described hereafter. Examples with and without psyllium were made using gelatin as the binding agent.

Examples 9 and 11-13 were made according to the following formulas.

| | Ex. 11 | Ex. 12 | Ex. 13 | Ex. 9 |
|---|---|---|---|---|
| | Psyllium Type | | | |
| | None | None | OT | XT |
| | (wt %) | (wt %) | (wt %) | (wt %) |
| Water | 14.00 | 10.76 | 11 | 9.81 |
| Corn Syrup 42 DE | 39.00 | 39.11 | 34 | 31.39 |
| Sucrose | 37.95 | 39.56 | 33.45 | 32.91 |
| Crisco ® Shortening¹ | 0 | 4.01 | 0 | 4.05 |
| Knox ® Gelatin | 7.00 | 4.51 | 2.5 | 2.54 |
| Citric Acid | 0.80 | 0.80 | 0.8 | 0.81 |
| Psyllium | 0 | 0 | 17 | 17.22 |
| Flavoring Agent | 1.20 | 1.20 | 1.2 | 1.22 |
| Coloring Agent | 0.05 | 0.05 | 0.05 | 0.05 |
| Total | 100.00 | 100.00 | 100.00 | 100.00 |
| kcal/100 g (psyllium: | 310 | 344 | 328 | 355 |
| 4 kcal/g) | | | | |
| % Solid content | 78.43 | 81.70 | 82.404 | 84.10 |
| (Theoretical) | | | | |
| % Moisture content | 21.57 | 18.30 | 17.596 | 15.90 |
| (Theoretical) | | | | |

¹Crisco ® Baking Sticks, Lot # 531342004 08:19 C.

The examples were held for 4 days at room temperature and about 60% RH before ejecting from the molds. The examples were stored in covered glass jars until texture parameters were measured. Texture Parameters were measured according to the methods described hereafter.

| | Ex. 11 | Ex. 12 | Ex. 13 | Ex. 9 |
|---|---|---|---|---|
| Mold Type: Polymer | | | | |
| Hardness Parameter | | 140 | | 2201 |
| (gf) | | | | |
| Adhesiveness | | −30.72 | | −857.05 |
| Parameter (gf s) | | | | |
| Springiness Parameter | | 0.89 | | 0.78 |
| Cohesiveness | | 0.78 | | 0.47 |
| Parameter | | | | |
| Gumminess Parameter | | 110 | | 1042 |
| (gf) | | | | |
| Water Activity (Aw) | | 0.67 | | 0.70 |
| Mold Type: Starch | | | | |
| Hardness Parameter | 1841 | 458 | 413 | 2713 |
| (gf) | | | | |
| Adhesiveness | −128.98 | −62.78 | −238.28 | −91.13 |
| Parameter (gf s) | | | | |
| Springiness Parameter | 0.91 | 0.95 | 0.98 | 0.53 |
| Cohesiveness | 0.95 | 0.71 | 0.65 | 0.42 |
| Parameter | | | | |
| Gumminess Parameter | 1755 | 324 | 268 | 1126 |
| (gf) | | | | |
| Water Activity (Aw) | 0.64 | 0.60 | 0.64 | 0.67 |

Examples 11 and 12 were made without the addition of psyllium and used as controls. Example 11 was a typical formula for commercially available soft chewable products and had a high level of gelatin. Example 11 had Hardness, Springiness, Gumminess and Cohesiveness Parameters that fell within the ranges known to be acceptable to consumers. Example 11 also shows that the Adhesiveness Parameter for a typical soft chewable product after 4 days is about −128 gf s. Example 12, which had a low level of gelatin and water and included shortening as a processing aid, had Hardness and Gumminess Parameters that fell below the ranges known to be acceptable to consumers.

It was surprisingly found that when psyllium having an initial particle size distribution of about 100% less than about 250 μm, about 92% less than about 212 μm, about 83% less than about 180 μm, about 61% less than about 150 μm, about 30% less than about 106 μm, and about 11% less than about 75 μm was incorporated into a formula with low gelatin, as in Example 9, the Hardness and Gumminess Parameters increased to levels that fell within the range known to be acceptable to consumers, while still having an acceptable Springiness Parameter. The adhesiveness also decreased with the addition of psyllium in Example 9 as compared to Example 11. However, when psyllium having an initial particle distribution of about 49.2% equal or less than about 710 μm and greater than about 250 μm was added to the formula, as in Example 13, psyllium did not act as a binder and the Hardness and Gumminess Parameters decreased to levels below the ranges known to be acceptable to consumers. The Hardness and Springiness Parameters of Example 13 were similar to Example 12, which did not contain psyllium.

It was found that the adhesiveness of the soft chewable compositions with psyllium were within the level known to be acceptable to consumers. When compared to a typical soft chewable product formula without psyllium at 4 days, the adhesiveness decreased when psyllium was added. Adhesiveness of the soft chewable composition can also be managed through post-processing steps described hereafter.

Alternative Binding Agents

Different formulas were tested to assess the impact of alternative binding agents on the texture of a soft chewable composition. Examples 14-17 were made according to the procedure described hereafter. The examples were made using psyllium XT and pectin or starch as the binding agent.

Examples 14-17 were made according to the following formulas.

| | Ex. 14 | Ex. 15 | Ex. 16 | Ex. 17 |
|---|---|---|---|---|
| | (wt %) | (wt %) | (wt %) | (wt %) |
| Water | 10.18 | 8.04 | 11.58 | 11.02 |
| Corn Syrup 42 DE | 30.75 | 29.20 | 24.26 | 35.67 |
| Sucrose | 31.75 | 29.55 | 25.63 | 28.95 |
| Crisco ® Shortening¹ | 4.03 | 4.10 | 3.96 | 4.14 |
| Knox ® Gelatin | 0 | 0 | 0 | 0 |
| Citric Acid | 0.81 | 0.83 | 0.81 | 0.83 |
| Psyllium | 17.14 | 25.62 | 33.66 | 17.58 |
| Flavoring Agent | 1.21 | 0 | 0 | 1.24 |
| Coloring Agent | 0.10 | 0.10 | 0.10 | 0.05 |
| PenBind ® 853 Starch² | 4.03 | 2.56 | 0 | 0 |
| Pectin LA-S20³ | 0 | 0 | 0 | 0.52 |
| Total | 100.00 | 100.00 | 100.00 | 100.00 |
| kcal/100 g (psyllium: | 356 | 366 | 355 | 349 |
| 4 kcal/g) | | | | |
| % Solid content | 83.85 | 86.29 | 83.71 | 82.06 |
| (Theoretical) | | | | |
| % Moisture content | 16.15 | 13.71 | 16.29 | 17.94 |
| (Theoretical) | | | | |

¹Crisco ® Baking Sticks, Lot # 531342004 08:19 C.
²Available from Ingredion, Inc., West Chester, IL
³Available PB Leiner, Plainview, NY

The examples were held for 4 days at room temperature and about 60% RH before ejecting from the molds. The examples were stored in covered glass jars until texture parameters were measured. Texture Parameters were measured according to the methods described hereafter.

| | Ex. 14 | Ex. 15 | Ex. 16 | Ex. 17 |
|---|---|---|---|---|
| Mold Type: Starch | | | | |
| Hardness Parameter | 1404 | 6290 | 15562 | 3395 |
| (gf) | | | | |
| Adhesiveness | −28.86 | −461.88 | −5.01 | −170.71 |
| Parameter (gf s) | | | | |
| Springiness | 0.48 | 0.57 | 0.61 | 0.57 |
| Parameter | | | | |
| Cohesiveness | 0.43 | 0.45 | 0.45 | 0.46 |
| Parameter | | | | |
| Gumminess | 606 | 2850 | 6980 | 1555 |
| Parameter (gf) | | | | |
| Water Activity (Aw) | 0.62 | 0.64 | 0.65 | 0.62 |

It was found that vegan and/or vegetarian soft chewable compositions could be made with texture parameters that fell within the ranges known to be acceptable to consumers.

It was found that gelatin can be replaced by a combination of starch and psyllium to obtain the binding properties and matrix formation needed to create an acceptable texture. Example 14, which had about 17% psyllium and 4% starch, had a low Hardness Parameter of about 1,404 gf and Gumminess Parameter of about 606 gf. However, in combination with a low Springiness Parameter of 0.48 and Cohesiveness Parameter of 0.43, the texture of Example 14 could still be consumer acceptable.

As the concentration of psyllium increased, the concentration of starch could be decreased because psyllium can act as a binder. The Hardness and Gumminess Parameters of Example 15, which had about 25% psyllium and 2.5% starch, increased to about 6,290 gf and 2,850 gf, respectively, but still fell within the ranges known to be acceptable to consumers. At high levels of psyllium, it was found that the binding agent could be removed from the formula and the corn syrup could be reduced. Example 16, which had about 34% psyllium and no binding agent, had a Hardness Parameter of about 15,562 gf and Gumminess Parameter of 6,980 gf, which are above the levels known to be acceptable to consumers, but the Springiness and Cohesiveness Parameters remained the same.

It was further found that gelatin can be replaced by a combination of pectin and psyllium. Example 17, which had about 17% psyllium and 0.5% pectin, had a Hardness Parameter of about 3,395 gf and Gumminess Parameter of about 1,555 gf, which fell within the ranges known to be acceptable to consumers. However, it can be challenging to obtain a consumer acceptable texture when formulating with psyllium and pectin. When psyllium levels are increased beyond about 20% in a pectin-based formula, a reduction of other ingredients, such as sucrose, is required for pectin to create an acceptable texture. Without being limited by theory, it is believed that it is difficult to formulate a soft chewable composition with pectin and psyllium because of the water competition between psyllium and pectin during the mixing and processing of the ingredients.

Low Water Formulas

Different formulas were tested to assess the impact of water in the formula on the ability to incorporate psyllium into a soft chewable composition and on the texture. Examples 18-21 were made according to the procedure described hereafter.

Examples 18-21 were made according to the following formulas.

| | Ex. 18 | Ex. 19 | Ex. 20 | Ex. 21 |
|---|---|---|---|---|
| | Psyllium Type | | | |
| | None | ST | OT | ST |
| | (wt %) | (wt %) | (wt %) | (wt %) |
| Water | 0 | 0 | 0 | 0 |
| Corn Syrup 42 DE | 35.00 | 30.00 | 30.00 | 29.70 |
| Crisco ® Shortening¹ | 6.00 | 6.00 | 6.00 | 6.00 |
| Citric Acid | 0.50 | 0.50 | 0.50 | 0.50 |
| Psyllium | 0 | 17.00 | 17.00 | 34.00 |
| Flavoring Agent | 1.20 | 1.20 | 1.20 | 1.20 |
| Coloring Agent | 0.05 | 0.05 | 0.05 | 0.10 |
| Glycerin | 2.00 | 5.00 | 5.00 | 10.00 |
| Confectionary Sugar | 55.25 | 40.00 | 40.00 | 18.25 |
| Lecithin | 0 | 0.25 | 0.25 | 0.25 |
| Total | 100.00 | 100.00 | 100.00 | 100.00 |
| kcal/100 g (psyllium: | 403 | 408 | 408 | 408 |
| 4 kcal/g) | | | | |
| % Solid content | 93.21 | 81.21 | 81.21 | 64.51 |
| (Theoretical) | | | | |
| % Moisture content | 6.65 | 10.5 | 10.5 | 10.395 |
| (Theoretical) | | | | |

¹Crisco ® Baking Sticks, Lot # 531342004 08:19 C.

The examples were held for 4 days at room temperature and about 60% RH before ejecting from the molds. The examples were stored in covered glass jars until texture parameters were measured. Texture Parameters were measured according to the methods described hereafter.

| | Ex. 18 | Ex. 19 | Ex. 20 | Ex. 21 |
|---|---|---|---|---|
| Hardness | 3261 | 3753 | 1685 | 5367 |
| Parameter (gf) | | | | |
| Adhesiveness | −105.79 | −412.17 | −491.65 | −299.98 |
| Parameter (gf s) | | | | |
| Springiness | 0.12 | 0.22 | 0.87 | 0.26 |
| Parameter | | | | |
| Cohesiveness | 0.10 | 0.15 | 0.36 | 0.16 |
| Parameter | | | | |
| Gumminess | 351 | 548 | 611 | 859 |
| Parameter (gf) | | | | |
| Water Activity | 0.60 | 0.55 | 0.55 | 0.48 |
| (Aw) | | | | |

It was found that psyllium can be incorporated into a low water soft chewable composition by adding lecithin and/or glycerin to the formula and replacing the sucrose with confectionary sugar, which has a finer particle size. Example 18, which did not contain psyllium, had a Hardness Parameter of about 3,261 gf, a Springiness Parameter of 0.12, and a Gumminess Parameter of about 351 gf. When psyllium with a particle size distribution of about 100% less than about 180 μm, about 98.4% less than about 150 μm, about 61.3% less than about 106 μm, and about 35.1% less than about 75 μm was added to the formula, as in Examples 19 and 21, the Hardness, Springiness and Cohesiveness Parameters increased. Examples 19 and 21 had texture parameters that fell within the ranges known to be acceptable by consumers and resulted in a composition with a soft texture that was slightly tacky and chewable, similar to the texture of a Starburst® soft chew. Example 20, which had coarser psyllium of an initial particle size distribution of about 49.2% equal or less than about 710 μm and greater than about 250 μm, had the lowest Hardness Parameter of only about 1,685 gf and highest Springiness Parameter of 0.87.

When the psyllium concentration was increased from about 17% to about 34%, as in Examples 19 and 21, the Hardness and the Gumminess Parameters increased, but still fell within the ranges known to be acceptable to consumers. In addition, the Springiness Parameter increased almost two fold as compared to the control when psyllium was added.

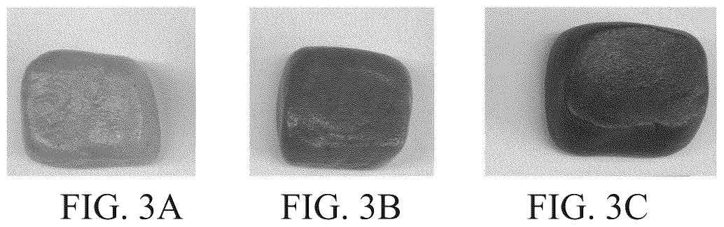

FIGS. 3A-3C show Examples 18, 19, and 21, respectively, to demonstrate the effect of increasing psyllium concentration on the appearance and texture of low water soft chewable compositions. It was found that as the psyllium concentration increased, the soft chewable composition had a darker appearance, but the psyllium particles were not visible.

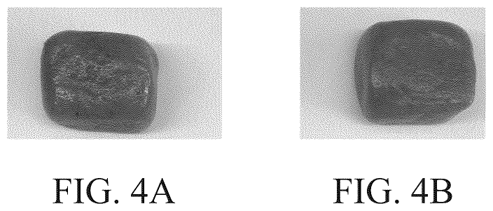

FIGS. 4A-4B show Examples 19 and 20 to demonstrate the effect of psyllium particle size on the appearance and texture of low water soft chewable compositions. Example 20, which had coarser initial psyllium particles, had a grainy appearance as compared to Example 19 made with finer psyllium particles.

The soft chewable composition can contain from about 1% to about 55% psyllium, alternatively from about 3% to about 45%, alternatively from about 5% to about 40%, alternatively from about 10% to about 35%, all by weight of the composition. The soft chewable composition can contain from about 1% to about 20% psyllium, alternatively from about 3% to about 15%, alternatively from about 7% to about 10%, all by weight of the composition. The soft chewable composition can contain from about 15% to about 50% psyllium, alternatively from about 20% to about 45%, alternatively from about 25% to about 40%, all by weight of the composition. In one aspect, the soft chewable composition can contain about 17% psyllium, by weight of the composition.

The soft chewable composition can comprise from about 1 g to about 45 g psyllium, alternatively from about 1.5 g to about 35 g, alternatively from about 1.7 g to about 17 g.

A single piece of the soft chewable composition can contain from about 1 g to about 15 g psyllium, alternatively from about 1.5 g to about 8 g psyllium, alternatively from about 1.5 g to about 6 g psyllium. A single piece of the soft chewable composition can contain about 1.7 g psyllium, alternatively about 2.5 g psyllium, alternatively about 3.4 g psyllium, alternatively about g psyllium, alternatively about 10.2 g psyllium.

As the level of psyllium increases, the hardness of the soft chewable composition can increase. The Hardness Parameter of the soft chewable composition can be from about 200 to about 20,000 gf, alternatively from about 400 to about 15,000 gf, alternatively from about 800 to about 10,000 gf, alternatively from about 1,000 to about 7,000 gf. The soft chewable composition can have a Hardness Parameter of from about 1,000 to about 10,000 gf, alternatively from about 2,000 to about 8,000 gf, alternatively from about 3,000 to about 6,000 gf.

The soft chewable composition can have a Hardness Parameter of greater than about 300 gf at a water activity of about 0.80 and less than about 10,000 gf at a water activity of about 0.50. It should be obvious to one skilled in the art that the Hardness Parameter for a given soft chewable composition changes monotonically as a function of water activity. The Hardness Parameter is measured according to the Texture Profile Analysis Method described herein. It is believed that if the Hardness Parameter of a soft chewable composition is too high, the texture may become unacceptable to consumers. If the Hardness Parameter is too low, the soft chewable composition may not be able to sustain its shape during storage and transportation.

As the level of psyllium increases, the cohesiveness of the soft chewable composition can decrease. The Cohesiveness Parameter of the soft chewable composition can be from about 0.30 to about 0.90, in another example about 0.40 to about 0.80, and in another example about 0.50 to about 0.75. The Cohesiveness Parameter is measured according to the Texture Profile Analysis Method described herein.

A change in the level of psyllium can cause a small change in the springiness of the soft chewable composition. Without being limited by theory, it is believed that this change can indicate that a more elastic structure can be created as the psyllium concentration increases in the soft chewable composition. The Springiness Parameter of the soft chewable composition can be from about 0.20 to about 0.95, alternatively from about 0.30 to about 0.85, alternatively from about 0.40 to about 0.80. The Springiness Parameter of the soft chewable composition can be greater than 0.50. The soft chewable composition can have a Springiness Parameter of greater than about 0.40 at a water activity of about 0.80 and less than about 0.80 at a water activity of about 0.50. It should be obvious to one skilled in the art that the Springiness Parameter for a given soft chewable composition changes monotonically as a function of water activity. The Springiness Parameter is measured according to the Texture Profile Analysis Method described herein.

The addition of psyllium into a soft chewable composition can increase the gumminess of the composition. As a result, the product can require more bites and more energy to be disintegrated in the mouth before swallowing, which consumers may find to be an acceptable texture for soft chewable products. The Gumminess Parameter of the soft chewable composition can be from about 100 gf to about 10,000 gf, alternatively from about 800 gf to about 8,000 gf, alternatively from about 1,000 gf to about 6,000 gf, alternatively from about 2,000 gf to about gf. The soft chewable composition can have a Gumminess Parameter of from about 300 gf to about 4,000 gf, alternatively from about 300 gf to about 1,000 gf. Alternatively, the soft chewable composition can have a Gumminess Parameter of from about 1,000 gf to about 5,000 gf. The Gumminess Parameter is measured according to the Texture Profile Analysis Method described herein.

Typical soft chewable products on the market can melt during storage, causing them to stick to each other and to the inside of containers. Consumers do not want products that melt together and/or leave a residue on the inside of the container because it can make a mess and make the product hard to handle and/or ingest. The addition of psyllium into a soft chewable composition can increase firmness and prevent melting of the soft chewable composition. One advantage to including psyllium in a soft chewable composition is that the composition may not easily melt or stick together.

The soft chewable composition can have an Adhesiveness Parameter of from about −1000 gf s to about 0 gf s, alternatively from about −500 gf s to about 0 gf s, alternatively from about −300 gf s to about 0 gf s, alternatively from about −100 gf s to about 0 gf s. The Adhesiveness Parameter is measured according to the Texture Profile Analysis Method described herein. It can be preferable to have the Adhesiveness value near zero so that the soft chewable compositions do not stick together or to teeth and gums during consumption.

The soft chewable composition can have a Hardness Parameter of less than about 6,000 gf, a Springiness Parameter of greater than about 0.50, and a Gumminess Parameter of less than about 6,000. One advantage to a soft chewable composition having these texture parameters is that it can provide a chewy structure that bounces back after biting or recovers the initial shape after deformation due to a stress.

The particle size distribution of psyllium can influence the appearance, overall texture, and mouthfeel of the soft chewable composition. If the initial particle size is too large, the particles may not fully disperse and/or dissolve. As a result, the particles can be visible in the soft chewable composition and the soft chewable composition can have a grainy mouthfeel.

The soft chewable composition can comprise psyllium having a particle size distribution as follows: about 100% less than about 250 μm, about 92% less than about 212 μm, about 83% less than about 180 μm, about 61% less than about 150 μm, about 30% less than about 106 μm, and about 11% less than about 75 μm. Alternatively, the soft chewable composition can comprise psyllium having a particle size distribution as follows: about 100% less than about 180 μm, about 98.4% less than about 150 μm, about 61.3% less than about 106 μm, and about 35.1% less than about 75 μm. Alternatively, the psyllium can comprise greater than about 80% of particles within the range of about 75 μm to about 250 μm. Alternatively, the psyllium can comprise greater than about 60% of particles within the range of about 75 μm to about 180 μm. Alternatively, the psyllium does not comprise particles greater than about 250 μm. Alternatively, the psyllium is substantially free of particles greater than about 250 μm. One advantage to using psyllium with an initial particle size within this range is that the particles can partially disperse and/or dissolve into the syrup mixture during processing to form a homogeneous slurry without causing a grainy feeling in the mouth. In one example, psyllium particles can be further ground during processing of the soft chewable composition and as a result, psyllium with a larger initial particle size can be used to form a soft chewable composition that has an acceptable texture.

The psyllium can be prehydrated before it is incorporated into the soft chewable composition. One advantage to prehydrating the psyllium is that it can prevent psyllium particles from getting stuck in a user's teeth or throat during consumption. The psyllium can be partially hydrated, alternatively completely hydrated, alternatively non-hydrated.

It has been found that partial pre-hydration and heating of psyllium at temperatures below about 120° C. during the production of the soft chewable composition does not disrupt the efficacy of psyllium. The various health benefits of psyllium can be attributed largely to its ability to form a viscous gel. Swell volume and water absorption index are two measures of psyllium gel formation, which are indirect methods of measuring efficacy.

The psyllium in the soft chewable composition can create a gel about as well as, if not better than, psyllium powder. Psyllium in the soft chewable composition have a swell volume greater than or equal to the swell volume of the psyllium before partial pre-hydration, heating, and cooling. The psyllium in the soft chewable composition have an average swell volume of from about 25 ml to about 50 ml, alternatively from about 30 ml to about 45 ml. The psyllium in the soft chewable composition have an average swell volume of about 43 ml. The average swell volume of psyllium before partial pre-hydration, heating, and cooling is about 30 ml. Without being limited by theory it is believed that other ingredients in the composition may contribute to the higher swell volume of the psyllium in the soft chewable composition, such as gelatin and/or starch, that can create a matrix by interacting with psyllium, which absorbs water and swell when hydrated. Swell volume is determined according to the Swell Volume Method described hereafter.

Psyllium in the soft chewable composition have a water absorption index (WAI) similar to the psyllium before partial pre-hydration, heating, and cooling. WAI is a quantitative measurement of how much water is absorbed by psyllium. Psyllium in the soft chewable composition can have a normalized WAI of from about 20 to about 60, alternatively from about 25 to about 50, alternatively from about 30 to about 45, alternatively from about 35 to about 41. WAI is determined according to the Water Absorption Index Method described hereafter.

The soft chewable composition can contain a binding agent. One advantage to using a binding agent is that it can give the soft chewable composition its plasticity, gumminess, chewy consistency, and texture. Another advantage to using a binding agent is that it can act as a structurant to form a network when water is removed. In one example, the soft chewable composition does not contain a binding agent because psyllium can act as a binder. In one example, the best texture can be obtained when a combination of psyllium and binding agent is utilized because of a synergistic effect. As the level of psyllium increases, the level of binder needed to form a soft chewable composition can decrease.

The soft chewable composition can contain gelatin. The soft chewable composition can comprise from about 0.1% to about 10% gelatin, alternatively from about 0.4% to about 6%, alternatively from about 0.8% to about 4%. One advantage to using gelatin is that it can provide elasticity and a chewy consistency. However, the texture of compositions formulated with gelatin can change with temperature during its shelf-life due to the fact that gelatin melts at temperatures around 35° C. One advantage to formulating a gelatin-containing soft chewable composition with psyllium is that it can delay the melting point of gelatin due to its ability to absorb water. In addition, it has been found that a soft chewable composition having psyllium can be formed with a gummy texture, even in the presence of low levels of gelatin. The natural gel formation that occurs when psyllium is hydrated can provide a gummy texture in a soft chewable composition that is comparable to traditional soft chewable products, eliminating the need for high levels of gelatin. Up to about 60% of gelatin in a soft chewable composition formula can be replaced with psyllium. Alternatively, the soft chewable composition can be substantially free of gelatin. The soft chewable composition can contain less than about 1%, alternatively less than about 0.05%, alternatively less than about 0.01% gelatin.

The soft chewable composition need not contain animal products and can be consumed on a vegan or vegetarian diet. The soft chewable composition can contain pectin. The soft chewable composition can comprise from about 0.01% to about 5% pectin by weight of the composition, in alternatively from about 0.1% to about 3%, alternatively from about 0.25% to about 1%. In one example, a soft chewable composition with a pectin base does not have greater than about 20% psyllium. In another example, a soft chewable composition with a pectin base does not have greater than about 30% psyllium. While not wishing to be bound by theory, it is believed that the strong interaction between pectin and other polysaccharides, such as psyllium, can reduce pectin's ability to create a strong gel. It is thought that in a low water formula, psyllium may absorb water faster than pectin. As a result, if psyllium levels in the formula are too high, pectin may remain in the syrup, acting as an inert ingredient.

The soft chewable composition can contain a starch. In one example, the starch can be a thin-boiling starch, which can be made from potato (such as PenBind® 853, sold by Ingredion, Inc., West Chester, IL); from tapioca (such as Purity Gum® 8 sold by Ingredion, Inc.); from sago (such as Elastigel® 1000) sold by Ingredion, Inc.); and combinations thereof. In one example, the starch can be a high amylose starch, such as Hi-Set® 377, Hylon® V and Hylon® VII, available from Ingredion, Inc.

The soft chewable composition can include high amylose starch, thin-boiling starch, psyllium, and combinations thereof. The soft chewable composition can include high amylose starch, thin-boiling starch, and psyllium at a ratio of about 30:40:30.

The soft chewable composition can contain from about 0.1% to about 10% starch by weight of the composition, alternatively from about 0.5% to about 8%, alternatively from about 1% to about 5%, alternatively from about 2% to about 4%. Alternatively, the soft chewable composition can comprise less than about 5% starch. Typically starches have a low gelling tendency and are not as useful in creating a chewy texture when used alone. As a result, starches are traditionally used in conjunction with gelatin to achieve the desired chewy texture for a chewy composition. However, it has been found that the combination of gelatinized starch and psyllium in a soft chewable composition can provide the gelling needed to create a chewy texture, without the need for gelatin. In one aspect, the soft chewable composition can contain starch and psyllium, but does not contain gelatin. In one aspect, the soft chewable composition does not contain starch.

The binding agent can be calcium salts (e.g. tricalcium phosphate, calcium carbonate, etc.). The soft chewable composition can comprise from about 1% to about 15% calcium salts, alternatively from about 5% to about 12%, alternatively from about 8% to about 10%.

The soft chewable composition can comprise less than about 15% tricalcium phosphate, alternatively less than about 10%, alternatively less than about 8%, alternatively less than about 5%. It was found that in some formulations, tricalcium phosphate can increase the hardness of the soft chewable composition. For instance, in one example, formulas with about 8% tricalcium phosphate can have a hardness that may be unacceptable to consumers. It is believed that tricalcium phosphate can act to control gelling. It was found that tricalcium phosphate can be at least partially replaced with calcium carbonate, sugar or a combination of syrups such as agave syrup and inulin to bring the hardness into a range that is acceptable to consumers.

The soft chewable composition can contain a processing aid. Non-limiting examples of processing aids can include, high melting point fats with a melting point in the range of about 30° C. to about 68° C. such as animal fats, fatty acids, saturated fats, Palmetic acid, and Stearic acid; Arachidic acid; hydrogenated plant oils such as palm oil; partially hydrogenated plant oils such as soybean oil and partially hydrogenated coconut oil; cocoa butter; fat substitutes such as olestra; emulsifiers including distilled monoglycerides such as Alphadim® 90 (available from Corbion, Lenexa, KS), polyglycerol esters, polysorbate 60, polysorbate 65, polysorbate 80, sorbitan monoestearate, lacto palmitate, diacetyl tartaric acid ester of mono- and diglycerides, acetylated monoglyceride, polyricinoleate, glyceron, modified lecithin, lecithin; and any other material that can limit the hydration of psyllium particles, and combinations thereof. One advantage to including a processing aid is that it can provide a partial or complete hydrophobic environment to control the hydration of the psyllium particles, and therefore slow down psyllium gelling and viscosity development in the syrup. This can be important to control the maximum filling time required during processing before the viscosity of the syrup becomes too high, making the molding step difficult. In one aspect, the addition of a processing aid to the psyllium can reduce the viscosity of the syrup.

The soft chewable composition can contain from about 0.01% to about 20% processing aid, alternatively from about 0.1% to about 15%, alternatively from about 0.20% to about 10%, alternatively from about 0.50% to about 8%. Alternatively, the soft chewable composition can include from about 0.01% to about 0.50% processing aid.

The soft chewable composition can comprise a blend of low melting point fat and high melting point fat. In one example, a low melting point fat can have a melting point of about −20° C. to about 30° C. Non-limiting examples of low melting point fat can include corn oil, canola oil, middle chain triglycerides, and combinations thereof. The blend of fats can comprise from about to about 50% high melting point fat. Alternatively, the soft chewable composition can comprise from about 0.5% to about 25% high melting point fat, alternatively about 0.5% to about 10% high melting point fat. The level of high melting point fat in the blend can depend on the melting point of the individual fats used to make the blend. Any level of high melting point fat and low melting point fat can be used to make the blend so long as the blend has a melting point in the range of about 30° C. to about 68° C., preferably from about 30° C. to about 55° C., more preferably from about 35° C. to about 45° C.

The soft chewable composition can contain a humectant component. The soft chewable composition can comprise from about 1% to about 40% of a humectant component, alternatively from about 3% to about 30%, alternatively from about 5% to about 25%, by weight of the composition. The soft chewable composition can comprise from about 20% to about 40% humectant component. Non-limiting examples of suitable humectant components can include glycerin, invert sugar, polyhydric alcohols, polyethylene glycol, propylene glycol, polyglycerol, xanthan gums, carageenans, alginates, cyclomethicone, sodium hyaluronate, sodium lactate, tracetin, triethanolamine, corn syrup, and mixtures thereof. One advantage to including a humectant component is that it can help form a soft chewable composition with a low moisture content that is still soft. Another advantage to including a humectant component is that it may help to reduce the viscosity of the syrup.

In one aspect, the soft chewable composition can comprise from about 3% to about 5%, by weight of the composition, glycerin.

The soft chewable composition can include a carbohydrate component. Non-limiting examples of suitable carbohydrate components include sucrose, polydextrose, trehalose, lactose, maltose, honey, glucose, galactose, confectionary sugar, maltodextrin, corn syrup solids, modified starches, and combinations thereof. The soft chewable composition can comprise from about 1% to about 55% carbohydrate component, alternatively from about 10% to about 45%, alternatively from about 20% to about 35%, by weight of the composition. The soft chewable composition can comprise from about 15% to about 55% carbohydrate component, alternatively from about 20% to about 40%, alternatively from about 25% to about 35%.

The soft chewable composition can be substantially free of an insoluble gum base which can comprise elastomers, polyvinylacetate, rubbers, chicle, jelutong, terpene resins, and combinations thereof.

The soft chewable composition can include a salt. Non-limiting examples of salts can include potassium chloride, sodium chloride, magnesium chloride, magnesium sulfate, and combinations thereof. The soft chewable composition can comprise from about 0.01% to about 10% salt, alternatively from about 0.1% to about 8%, alternatively from about 0.5% to about 5%, alternatively about 1% to about 2%, by weight of the composition. The soft chewable composition can comprise about 0.1% to about 1% salt. The soft chewable composition can comprise about salt. In some cases, if the level of salt is greater than about 5%, the soft chewable composition can have a salty taste when consumed. One advantage to including a salt is that it can help reduce the viscosity of the syrup.

The soft chewable composition can include a sweetener. Non-limiting examples of sweeteners can include stevia, monk fruit sugar, agave syrup, crystalline fructose, high fructose corn syrup, tapioca syrups, sucralose, aspartame, neotame, sorbitol, xylitol, saccharin, cyclamate, and combinations thereof. The soft chewable composition can include natural or artificial sweeteners, sugar alcohol, or other sugar substitute in place of all or part of its sucrose. The soft chewable composition can be sugar-free. The soft chewable composition can comprise from about to about 1% sucralose, alternatively from about 0.01% to about 0.5%, alternatively from about 0.03% to about 0.1%.

The soft chewable composition can include a preservative. Non-limiting examples of suitable preservatives can include: potassium sorbate, sodium benzoate, sodium citrate, sodium phosphate, potassium metabisulfite, sodium metabisulfite, sodium lactate, sodium sulfite, ethylenediaminetetraacetic acid (EDTA), methylparaben, and mixtures thereof. The soft chewable composition can include from about 10 to about 100 ppm preservative, alternatively from about 20 to about 80 ppm, alternatively from about 30 to about 50 ppm. The preservative can be an antioxidant. One advantage to including an antioxidant in the soft chewable composition is that it can help to control fat oxidation. Non-limiting examples of suitable antioxidants can include tocopherols, rosemary extract, butylated hydroxytoluene, and combinations thereof.

To balance flavor and regulate the pH of the soft chewable composition, food grade acid can be added to the syrup during processing. The pH of the syrup can be from about 3 to about 4.5. One advantage to having a pH in this range is that it can preserve the soft chewable composition and help with microbial growth stability. Non-limiting examples of such food acids can include citric acid, malic acid, lactic acid, adipic acid, fumaric acid, tartaric acid, phosphoric acid, mono-potassium phosphate, any other suitable food grade acid, and combinations thereof. The soft chewable composition can comprise from about 0.5% to about 4% citric acid, alternatively from about 1% to about 3.5%, alternatively from about 1.5% to about 3%.

In one aspect, the addition of a food grade acid to the soft chewable composition can also help to control the swelling of the psyllium in the mouth.

The soft chewable composition can include a flavoring agent. Non-limiting examples of flavors can include natural or artificial flavors such as chocolate; vanilla; caramel; coffee; fruit flavors including lemon, lime, orange, blackberry, raspberry, blueberry, peach, apricot, cherry, and grape; and mixtures thereof. The soft chewable composition can include from about 0.001% to about 7% flavoring agent, alternatively from about 0.01% to about 5%, alternatively from about to about 3%, alternatively from about 0.5% to about 1.5%.

The soft chewable composition can include a coloring agent. Coloring agents can be added to the soft chewable composition to achieve the desired color, including: red dye #40; yellow dye #5; yellow dye #6; blue dye #1, and combinations thereof. Color additives may also include natural coloring such as black carrot, annatto, tumeric, paprika, fruit and vegetable concentrated juices (e.g. purple berry concentrate), and combinations thereof. The soft chewable composition can include from about 0.001% to 5%, alternatively from about 0.01% to about 3%, alternatively from about 0.05% to about 1%. As the amount of psyllium increases in the formula, the color of the soft chewable composition can become darker and less coloring agent is needed.

The soft chewable composition can also include a supplement component including, but not limited to, vitamins, minerals, herbs, botanicals, plant derived supplements, therapeutic compounds, and mixtures thereof.

Non-limiting examples of such supplemental components include: potassium, B vitamins, vitamins A, C, D, E, and K, folic acid, other vitamins and minerals commonly known in the art and used for supplementing the diet, amino acids, extracts and active phytochemicals including ferulic acid (from apples), ginseng, ginko biloba, beta carotene, capsicanoids, anthocyanidins, bioflavinoids, d-limonene, isothiocyanates, cysteines from garlic, ginger, grapes, catechins and polyphenols from teas, onions, phytosterols, isoflavones, lycopene, curcumin, caffeine, glucosamine, chondroitin, melatonin, omega-3 fatty acids, serotonin, probiotics, prebiotics, and mixtures thereof.

The soft chewable composition can comprise from about 0.001% to about 25%, alternatively from about 0.01% to about 15%, alternatively from about 0.1% to about 5%, by weight of the composition, of a supplement component.

The soft chewable composition can contain an active ingredient such as metformin, statins, sodium carbonate, magnesium carbonate, H2 antagonists, magnesium hydroxide, aluminum hydroxide, omeprazole, pantoprazole, lansoprazole, bismuth subsalicylate and combinations thereof.

The soft chewable composition can contain additional dietary fibers. Non-limiting examples of insoluble fibers can include wheat bran and cellulose. Non-limiting examples of soluble fiber can include inulin, soluble corn fiber, soluble tapioca fiber, beta-glucan, partially hydrolyzed guar gum, wheat dextrin, acacia, galacto-oligosaccharides, fructo-oligosaccharides, or xylo-oligosaccharides. The soft chewable composition can comprise from about 1% to about 80%, alternatively from about 15 to about 60%, alternatively from about 30 to about 50%, by weight of the composition, of an additional dietary fiber. The soft chewable composition can comprise from about 1% to about 40% additional dietary fiber, alternatively from about 5% to about 35%, a alternatively from about 10% to about 25%. The soft chewable composition can comprise from about 40% to about 80% additional dietary fiber, alternatively about 50% to about 60%.

The soft chewable composition can be center-filled with a liquid, syrup, or powder. The center filling can contain vitamins, supplements, nutritional ingredients, minerals, herbal extracts, flavoring, additional dietary fiber, chocolate or other forms of confectionary products, and the like.

The soft chewable composition can be coated. The coating can be comprised of linsic oil, bees wax, carnauba wax, or any other suitable food grade oil, sucrose, sugar alcohol ingredients, or combinations thereof. The coating can also be comprised of chocolate, white chocolate, or other dairy or non-dairy fat based food approved ingredients.

The final moisture content of the soft chewable composition can impact the texture of the soft chewable composition. The soft chewable composition can have a finished moisture content of about 5% to about 25%, alternatively from about 10% to about 23%, alternatively from about 13% to about 21%.

The soft chewable composition can have an Aw at the time of production of about 0.45 to about 0.85, alternatively from about 0.55 to about 0.75, alternatively from about 0.60 to about 0.70. The Aw of the soft chewable composition at the time of production can impact the springiness and the gumminess of the composition. As the Aw at the time of production increases, the springiness and cohesiveness of the soft chewable composition also increases.

The Aw at the time of production can be adjusted to achieve the desired Aw of the final product. The Aw at the time of production should not be higher than about 0.78 if the soft chewable composition will be molded in a starch mold because during curing the Aw can drop to about 0.2 Aw. It was found that starch molds can absorb water and impact the texture of the final soft chewable composition. The Aw at the time of production should be from about 0.70 to about 0.75 if the soft chewable composition will be molded in a non-absorbent mold, such as a polymer mold.

For soft chewable compositions, the Aw of the product can be important to predict the shelf life. Soft chewable compositions are considered intermediate moisture content products, and as a result, one of the key quality concerns is microbial growth. At an Aw greater than about 0.70, mold can grow on the surface of the product over its shelf life. Soft chewable products with an Aw less than about 0.50 can have a hardness that can be unacceptable to consumers. Therefore, it is important to balance the optimum Aw of the finished product to obtain micro stability with hardness.

The soft chewable composition, as packaged, can have an Aw of from about 0.50 to about alternatively from about 0.60 to about 0.76, alternatively from about 0.65 to about 0.74. One advantage to an Aw in this range is that it can provide stability against the growth of mold. When the Aw is greater than about 0.80 and formula can include a preservative to provide stability and/or prevent mold growth. One advantage to having an Aw greater than about 0.80 is that it can provide a softer texture. Water Activity is determined as described hereafter in the Water Activity Test Method described hereafter.

The Aw of the soft chewable composition as packaged can be controlled by the type of molding used during processing, by adjusting the finished percent of solids in the formula, and/or the storage conditions. The percent solids in the formula can be from about 70% to about 85% alternatively from about 75% to about 83%, alternatively from about 77% to about 80%. The percent solids of the soft chewable composition can be controlled by heating to boiling during processing. Alternatively, in some soft chewable compositions, the formulation can be designed to target a desired percent solids, such as in the low water formulations.

The soft chewable composition can have a shelf life of at least about 12 months, alternatively at least about 18 months, alternatively at least about 24 months.

The methods herein may comprise orally administering a dose of about 1 to about 10, or about 1 to about 6, or about 1 to about 4, or about 1 to about 2, pieces of a soft chewable composition per day. The compositions may comprise at least about 17% of psyllium, by weight of the composition.

In one example, if a user wished to ingest about 10.2 grams of psyllium per day, the user could ingest 1 piece per day comprising about 10.2 grams of psyllium, alternatively the user could ingest 2 pieces per day comprising about 5.1 grams of psyllium each, alternatively the user could ingest 3 pieces per day comprising about 3.4 grams of psyllium each, alternatively the user could ingest 4 pieces comprising about 2.5 grams of psyllium each, alternatively the user could ingest 6 pieces per day comprising about 1.7 grams of psyllium each.

The soft chewable composition can be consumed one time per day or multiple times per day. The soft chewable composition can be consumed twice per day. Alternatively, the soft chewable composition can be consumed three times per day. The soft chewable composition can be consumed on a daily basis or only as needed. In one example, the soft chewable composition can be taken about 30 minutes, about 60 minutes, about 90 minutes, or about 120 minutes after eating. The soft chewable composition can be taken on an empty stomach or with food. The soft chewable composition can be taken before or with meals to help with appetite control and/or blood glucose control. Alternatively, the soft chewable composition can be taken about three times per day, before or after meals, to help with digestive wellness and/or heart health benefits. The soft chewable composition can be taken without water. Alternatively, the soft chewable composition can be taken with about 8 ounces of water.

Another aspect of the present invention includes methods of providing one or more health benefits comprising orally administering the present composition to a user. As used herein, the one or more health benefits may be selected from the group consisting of providing digestive wellness, providing fiber; laxation; increased stool volume and moisture content; intestinal regularity; slowed gastrointestinal transition and digestion processes; modified fat absorption; weight management; increasing satiety; increasing excretion of bile acids; benefiting the postprandial glycemic response; controlling blood glucose; aiding growth and/or development of beneficial gastrointestinal microorganisms; promoting hearth health; lowering blood cholesterol; as well as reduce the risk of heart disease, diabetes, obesity, and/or colon cancer, and any combination of the foregoing. In one embodiment herein, the one or more health benefits may be selected from the group consisting of providing digestive wellness; fiber; laxation; increased stool volume and moisture content; intestinal regularity; slowed gastrointestinal transition and digestion processes; modified fat absorption; aiding in weight management; increasing excretion of bile acids benefiting the postprandial glycemic response; aiding growth and/or development of beneficial gastrointestinal microorganisms, and any combination of the foregoing. In one embodiment herein, the one or more health benefits may be selected from the group consisting of providing digestive wellness, providing fiber, laxation, and any combination of the foregoing. In one embodiment herein, the one or more health benefits may be selected from the group consisting of promoting hearth health, lowering blood cholesterol, reduce the risk of heart disease, and a combination of the foregoing. In one embodiment herein, the one or more health benefits may be selected from the group consisting of increasing satiety, weight management, reducing the risk of diabetes, reducing the risk of obesity, controlling blood glucose, and any combination of the foregoing.

The glucose diffusion pattern of the psyllium in the soft chewable composition is similar to the glucose diffusion pattern of dry powder psyllium sold as Metamucil® (distributed by the Procter & Gamble Co., Cincinnati, Ohio), as measured by in vitro methods, which indicates that the psyllium in the soft chewable composition can deliver a similar impact on controlling blood glucose as well as powder psyllium. In an in vitro Glucose Diffusion Study, the psyllium in the soft chewable composition reduced glucose diffusion by a range of about 2% to about 7% as compared to control without psyllium. The Glucose Diffusion Study can be performed as described in Zacherl et al., In vitro model to correlate viscosity and bile acid-binding capacity of digested water-soluble and insoluble dietary fibres, 126 Food Chemistry 423-428 (2011), incorporated herein by reference. In particular, a soft chewable composition test sample is digested in a static digestion model that simulates the conditions of the mouth, stomach and duodenum as described in Zacherl et al. Next, the digested extract is mixed with a known amount of glucose and aliquoted into dialysis tubing. Suitable dialysis tubing can include Spectra/Por® 16 mm diameter dialysis tubing with a MW cutoff of 12,000-14,000 (available from Spectrum® Labs, Rancho Dominguez, CA). The filled dialysis tubes are placed in bottles of water containing a glass marble and shaken in a 37° C. water bath at 100 rpm to simulate mechanical peristaltic action of the small intestine. At 0.25, 0.5, 1 and 2 hours, samples of water surrounding the dialysis tubes are taken and glucose concentrations are measured using a commercial kit.

It can take from about 3 to about 25 chews before the soft chewable composition is ready for swallowing, alternatively from about 5 to about 15 chews, alternatively from about 10 to about 12 chews.

The soft chewable composition can contain from about 300 to about 450 kcal per 100 g, alternatively from about 340 to about 410 kcal per 100 g. The amount of calories can be calculated by considering psyllium as part of the total carbohydrates in the formula, even though it is a non-digestible carbohydrate, and using a caloric contribution factor of 4 kcal/g.

The soft chewable composition can be a reduced sugar formulation. As used herein, a reduced sugar formulation can comprise less than 50% sugar, alternatively less than 40% sugar, alternatively less than 20% sugar, alternatively less than 15% sugar. Reduced sugar formulations of the soft chewable compositions can be formulated using dry fructose, polyols, sugar alcohols such as isomalt, or oligosaccharides like inulin to at least partially replace sucrose and/or corn syrup.

Method of Making a Soft Chewable Composition

The present invention also relates to processes for making a soft chewable composition containing psyllium.

In one example, a method of preparing a soft chewable composition, wherein the soft chewable composition is a gummy, can comprise the steps of:
a. adding a binding agent to a first mixing vessel;
b. pre-treating the binding agent;
c. adding a humectant component and water to a second mixing vessel and mixing while heating to a temperature of from about 65° C. to about 72° C.;
d. adding a carbohydrate to the second mixing vessel to form a syrup pre-mixture and mixing while heating to form a cooked syrup pre-mixture;
e. adding the pre-treated binding agent to the second vessel and mixing while heating to form a base syrup mixture;
f. adding a processing aid to the base syrup mixture and mixing while heating;
g. adding a psyllium mixture to the base syrup mixture and mixing to form a final mixture;
h. optionally heating the final mixture to a temperature sufficient to achieve a desired solids content;
i. forming the final mixture into a soft chewable composition by molding;
j. cooling and optionally curing the soft chewable composition; and
k. optionally post-processing the soft chewable composition.

The syrup pre-mixture can be heated to a temperature of about 93° C. to about 177° C. The syrup pre-mixture can be heated to a temperature of about 113° C.

The pre-treatment step can vary depending on the binding agent used in the formula. In the case of a gelatin binding agent, pre-treating can comprise of hydrating the gelatin by adding water to the gelatin at a ratio of about 2:1 to about 3:1 and mixing at room temperature until the gelatin is completely hydrated. In the case of a starch binding agent, pre-treating can comprise of gelatinizing the starch by adding water to the starch and heating while mixing to a temperature of about 77° C. until the color of the starch binding agent changes from opaque white to clear grey. In the case of a pectin binding agent, pre-treating can comprise of mixing sucrose with the pectin to create a pectin-sucrose mix.

Additional ingredients, such as coloring agents, flavoring agents, processing aids, salts, food grade acids, supplement components, active ingredients, and combinations thereof, can be added to the cooked syrup pre-mixture. One advantage to adding the additional ingredients to the cooked syrup pre-mixture is that these ingredients may be temperature sensitive. The additional ingredients can be added to the base syrup mixture. Alternatively, the additional ingredients can be added to the base syrup mixture after adding the processing aid. Alternatively, the additional ingredients can be added to the final mixture. One advantage to adding the additional ingredients to the final mixture is that it can help to delay hydration and/or aid in processability.

The processing aid can first be heated in a separate mixing vessel to a temperature above its melting point before it is added to the base syrup mixture. The processing aid can be shortening and can be heated to a temperature greater than about 47° C. Alternatively, the processing aid can be separately melted before it is added to the base syrup mixture.

A psyllium mixture can comprise psyllium. Alternatively, a psyllium mixture can comprise psyllium and additional ingredients. A psyllium mixture can be prepared by mixing psyllium with the additional ingredients before it is mixed with the processing aid. A psyllium mixture can be formed by combining psyllium, citric acid, a flavoring agent, and a coloring agent. Alternatively, the psyllium mixture can be mixed with the processing aid before it is added to the base syrup mixture. A salt can also be added to the psyllium mixture before it is added to the base syrup mixture.

The psyllium mixture can be added to the base syrup mixture just prior to molding to prevent a significant increase in viscosity. It was found that increasing the temperature of psyllium up to about 95° C. significantly reduces the viscosity. However, this increase in temperature can also increase the hydration rate of psyllium particles, resulting in an increased viscosity again after 15 minutes. The final mixture can be processed in a mold or extruded within about 15 minutes of adding psyllium. Adding citric acid and/or salt to the psyllium mixture before it is added to the base syrup mixture can help to reduce the viscosity of the syrup and can increase the time for molding and/or extrusion to about 20 minutes, alternatively about 30 minutes, alternatively about minutes, alternatively about 90 minutes.

The final mixture can be mixed for about 5 minutes to about 60 minutes, alternatively for about 10 minutes to about 50 minutes, alternatively for about 15 minutes to about 40 minutes, alternatively for about 20 minutes to about 30 minutes. One advantage to mixing the final mixture is that it can reduce the viscosity of the final mixture and increase the time for molding and/or extrusion. After the final mixture has started gelling, physical sheer, such as mixing or pumping, can be used to break up the gel structure and lower the viscosity.

The psyllium can be agglomerated with an agglomerating material. The agglomerating materials useful herein are known, having been described in detail in U.S. Pat. No. 5,340,580 to Barbera, and U.S. Pat. Nos. 4,548,806 and 4,459,280, both to Colliopoulos et al., the disclosures of which are incorporated herein by reference in their entirety. These agglomerating materials are selected from the group consisting of water dispersible hydrolyzed starch oligosaccharide, mono-saccharide, di-saccharide, polyglucose, polymaltose, and mixtures thereof. The agglomerating material can include sucrose, salt, acid, maltodextrin, and combinations thereof. The soft chewable composition can comprise from about 0.5% to about 20% of agglomerating material coating on the psyllium, alternatively from about 1% to about 10%, alternatively from about 1% to about 5%.

The psyllium can be agglomerated before it is added to the base syrup mixture. The agglomeration process can comprise the steps of (a) coating to agglomerate a psyllium-containing blend, preferably a dry blend, with a solution mixture comprising one or more agglomerating materials; (b) drying the agglomerated psyllium; and (c) optionally, repeating steps (a) and (b). Step (c) is only optional, however, if one coating and drying step is sufficient to uniformly disperse at least about 0.5% of the acid throughout the agglomerating material coating on the psyllium, otherwise it is necessary to repeat steps (a) and (b) at least as many times as necessary to attain at least this level of acid uniformly dispersed.

Agglomeration techniques are described in the hereinbefore referenced U.S. patents. In one example, a multiple layer coating is applied to the psyllium using techniques which result in agglomerating the psyllium, e.g., as described in detail in U.S. Pat. Nos. 4,459,280 and 4,548,806, to Colliopoulos et al., incorporated by reference herein, is used. In another example, an agglomerating material (especially maltodextrin) is applied as a single coating in a single pass apparatus such that from about 5% to about 20% of water is applied to the psyllium husk during the coating process is used.

Multiple layer coating of the psyllium is accomplished, for example, by using fluid bed agglomerating equipment. An example of such fluid bed agglomerating equipment is the Fluid Air, Inc., Model 0300 Granulator-Dryer (sold by Fluid Air, Inc., Aurora, Illinois). Single layer coating of the psyllium is achieved by utilizing equipment which operates preferably by dropping a dry blend psyllium-containing material through a highly turbulent annular zone formed by a cylindrical wall and a rotating shaft with variously pitched attached blades. An agglomerating material-containing solution, is sprayed into this zone to contact the dry psyllium-containing blend. The resulting coating psyllium is dropped to a fluid bed dryer where the added solvent is removed. An example of this equipment is the Bepex Turboflex Model No. TFX-4 (sold by Bepex Corporation; Minneapolis, Minn.) with a six square foot bed vibrating fluid bed dryer (sold by Witte Corporation, Inc., Washington, N.J.).

The psyllium can be blended with about 70% sucrose and then sprayed with a 40% solution of citric acid followed by fluid bed drying.

One advantage to agglomerating the psyllium before adding it to the base syrup mixture is that it can help to delay the hydration of psyllium and can help to lower viscosity. Another advantage is that it can help improve the mouthfeel of the soft chewable composition. It is believed that the use of agglomerated psyllium can increase the dissolution rate of psyllium in the mouth, thereby reducing mouth dryness, and can reduce the gelling of psyllium in the mouth.

Metamucil® Smooth Texture Sugar Orange (distributed by the Procter & Gamble Co., Cincinnati, Ohio) can be used as the source of psyllium, sugar and/or citric acid. Metamucil® Smooth Texture Sugar Orange can be added to the base syrup mixture. One advantage to using Metamucil® Smooth Texture Sugar Orange as the source of psyllium is that it can slow the gelling of the psyllium and therefore control viscosity development when mixed with water and/or syrup during processing. This can improve processability of the soft chewable composition.

Alternatively, the psyllium can be unagglomerated. The humectant, carbohydrate, and water can be combined in the second mixing vessel and heated to a temperature of about 113° C. to form a cooked syrup pre-mixture. The pre-treated binding agent can then be added to the cooked syrup pre-mixture to form the base syrup mixture.

The final mixture can be formed into a soft chewable composition by molding or extrusion. The final mixture can be poured into a starch mold, via the Mogul process, or in a non-absorbent mold to create a soft chewable composition. Non-limiting examples of non-absorbent molds can include polymer, glass, metal, plastic, polytetrafluoroethylene, and any other material that does not absorb moisture.

The final mixture can be poured into a starch mold and allowed to cure. The starch mold can be any shape that is created by printing on the surface of the starch using a metallic board. The final mixture can be poured into a sheet mold to create a sheet of the soft chewable composition. The sheet can be cut into individual pieces and placed into starch molds to cure. The individual cut pieces can be placed in a tumbling drum with starch and continuously mixed for the time needed to increase the solids content to about 70 to about 80%. In one example, the soft chewable composition can cure for about 1 day to about 5 days before packaging. The soft chewable composition can cure for about 4 days, in another example for about 2 days, and in another example about 1 day before packaging. The curing time can be reduced by filling the mold with a final mixture that is at the target solids content. The soft chewable composition can be cured at room temperature, alternatively the soft chewable composition can be cured at about 22° C. to about 60° C. The soft chewable composition can be cured in a curing room with about 15% to about 25% RH.

Alternatively, the final mixture can be poured into a non-absorbent mold and allowed to cool. When using a non-absorbent mold, the solids concentration of the final mixture can be close to the desired finished product solids level because no significant changes in moisture will occur. The non-absorbent mold can provide the shape of the soft chewable composition, alternatively the soft chewable composition can be cut into the desired shape after it is removed from the non-absorbent mold. The soft chewable composition can be placed in a refrigerator and cooled to a temperature of about 20° C. to about 40° C.

The soft chewable composition can optionally be post-processed to decrease curing time, control texture such as adhesiveness, improve taste, improve stability, improve processability, and/or facilitate the dosing of the psyllium. Post-processing can include cutting, drying, individually wrapping the soft chewable composition pieces, dusting the soft chewable composition with sugar or starch after removal from the mold, coating the soft chewable composition after removal from the mold, leaving the soft chewable composition in the mold until the desired adhesiveness is achieved, enrobing, coextrusion, and combinations thereof. One advantage to post-processing the soft chewable composition is that it can prevent individual pieces of the soft chewable composition from sticking together during packaging and can make them feel less sticky during handling. Another advantage to post-processing is that it can reduce viscosity and increase time for molding and/or extrusion which can improve processability.

In one example, a method of preparing a low water soft chewable composition can comprise the steps of:
a. adding a humectant component to a first mixing vessel;
b. mixing while heating the first mixing vessel to a temperature of from about 65° C. to about 71° C.;
c. adding a carbohydrate to the first mixing vessel to form a humectant-syrup pre-mix and mixing while heating to form a cooked humectant-syrup mixture;
d. mixing a processing aid and a psyllium mixture in a third mixing vessel to form a psyllium-processing aid mixture;
e. adding the psyllium-processing aid mixture to the cooked humectant-syrup mixture and mixing while heating to form the final dough;
f. forming the final dough into the soft chewable composition with extrusion dies or wire cutting; and
g. optionally post-processing the soft chewable composition.

The psyllium mixture can be added to the cooked humectant-syrup mixture to form a final dough and extruded to form the soft chewable composition in the desired shape and size. Alternatively, the final dough can be spread into a tray and cut into pieces. The soft chewable composition can be cooled to a temperature of about 20° C. to about 40° C. before packaging.

It should be understood that the formulation for the soft chewable composition can be designed to achieve a specific final solids content without the need for heat to temperatures above the boiling point to evaporate solids using high levels of plasticizers such as oils and emulsifiers. Alternatively, the formulation for the soft chewable composition can be designed such that boiling is required during processing to achieve the desired solids content.

The soft chewable composition can be formed into any suitable convenient, ingestible form. Non-limiting examples of the form of the compositions include: soft chew, hard chew, soft gel, semi-solid taffy-like chew, gummies, and combinations thereof. The soft chewable composition can be in the form of a single piece of soft chew or a single piece of gummy. The soft chewable composition can be in a partitionable form, such as a bar, which the user can cut or break to provide individual pieces. A piece of the soft chewable composition can be from about 500 to about 7000 mm³, alternatively from about 1000 to about 5000 mm³, alternatively from about 1500 to about 4000 mm³. A piece of the soft chewable composition can have a volume of about 100,000 mm 3 and can be broken into smaller pieces. The soft chewable composition can be formed into any shape and size as long as it provides a volume within this range. Non-limiting examples of shapes can include circles, squares, rectangles, stars, hearts, animal shapes, and combinations thereof.

The soft chewable compositions can be packaged in any suitable package. The soft chewable composition can be individually wrapped in food grade packaging. The soft chewable composition can be individually wrapped and packaged together with enough pieces for a single dose, alternatively enough for a daily dose. Non-limiting examples of food grade packaging can include monoaxially oriented polypropylene, poly-lined foil wrappers, foil, and combinations thereof. Alternatively, the soft chewable composition can be unwrapped.

The soft chewable composition can be placed in secondary packaging, non-limiting examples of which include glass bottles; plastic bottles; foil lined bags, foil lined containers, cartons, or sleeves; and combinations thereof. The soft chewable composition can be packaged as single doses so they are easily portable and can be carried in a purse, pocket, or brief case. The packaging can be child resistant. The packaging can be transparent, alternatively the packaging can be opaque. The package can include a desiccant. The secondary packaging can contain an ultraviolet (UV) inhibitor because the soft chewable composition can be light sensitive. Alternatively, the secondary packaging does not contain a UV-inhibitor. The secondary packaging can contain a water and/or oxygen barrier because the soft chewable composition can be water and/or oxygen sensitive. Alternatively, the secondary packaging does not contain a water and/or oxygen barrier.

Gelatin based soft chewable compositions (Examples 1-13) were made according to the following procedure.

First, the gelatin was pre-treated to hydrate the gelatin. In a first mixing vessel, water was added to the gelatin at a ratio of 2:1 and mixed at room temperature until the gelatin was completely hydrated.

Second, corn syrup was diluted in water in a second mixing vessel. The second mixing vessel was heated using a hot plate while continuously stirring to 66-72° C. Then sucrose was slowly added to the second mixing vessel to form a syrup pre-mixture and heated with agitation to a temperature of 113±5° C. until the solids content reached greater than about 75% by weight of the syrup pre-mixture, resulting in a cooked syrup pre-mixture. Then, the pre-treated gelatin was added to the cooked syrup pre-mixture and mixed until complete dispersion was achieved, resulting in a base syrup mixture.

Simultaneously, shortening was separately heated to above its melting point of about 47° C. The melted shortening was added to the hot base syrup mixture and mixed until the shortening was incorporated into the base syrup mixture.

A psyllium mixture was separately made by mixing the psyllium, citric acid, coloring agent, and flavoring agent. The psyllium mixture was then added to the base syrup mixture to create a final mixture and mixed until homogenous.

To form the soft chewable composition, the final mixture was poured into a starch mold or a polymer mold and allowed to cool and/or cure before ejecting from the mold.

Starch based soft chewable compositions (Examples 14-16) were made according to the following procedure.

First, the starch was pre-treated to gelatinize the starch. In a first mixing vessel, water was mixed with starch at a ratio of 1:10 (starch:water) and heated with gentle stirring to 77±5° C. until the color of the starch solution changed from opaque white to clear grey.

Second, corn syrup was diluted in water in a second mixing vessel. The second mixing vessel was heated using a hot plate while continuously stirring to 66-72° C. Then sucrose was slowly added to the second mixing vessel to form a syrup pre-mixture and heated with agitation to a temperature of 113±5° C. until the solids content reached greater than about 75% by weight of the syrup pre-mixture, resulting in a cooked syrup pre-mixture. Then, the pre-treated starch was added to the cooked syrup pre-mixture and mixed until complete dispersion was achieved, resulting in a base syrup mixture.

Simultaneously, shortening was separately heated to above its melting point of 47° C. Psyllium was then blended with the melted shortening and added to the base syrup mixture to create a final mixture. The remaining ingredients (citric acid, coloring agent, and flavoring agent) were then added to the final mixture and mixed until homogenous.

To form the soft chewable composition, the final mixture was poured into a starch mold or a polymer mold and allowed to cool and/or cure before ejecting.

A pectin based soft chewable composition (Example 17) was made according to the following procedure.

First, the pectin was pre-treated in a first mixing vessel by blending sucrose with the pectin to create a pectin-sucrose mixture. Second, corn syrup was diluted in water in a second mixing vessel. The second mixing vessel was heated using a hot plate while continuously stirring to 66-72° C. Then the pectin-sucrose mixture was slowly added to the second mixing vessel to form a syrup pre-mixture and heated with agitation to a temperature of 113±5° C. until the solids content reached greater than about 75% by weight of the syrup pre-mixture, resulting in a cooked syrup pre-mixture.

Simultaneously, shortening was separately heated to above its melting point of 47° C. Psyllium was then blended with the melted shortening and added to the base syrup mixture to create a final mixture. The remaining ingredients (citric acid, coloring agent, and flavoring agent) were then added to the final mixture and mixed until homogenous.

To form the soft chewable composition, the final mixture was poured into a starch mold or a polymer mold allowed to cool and/or cure before ejecting.

Low water soft chewable compositions (Examples 18-21) were made according to the following procedure.

First, a humectant-syrup pre-mix was made by blending corn syrup with glycerin. The humectant-syrup pre-mix was heated using a hot plate while continuously stirring to 60-72° C. Then confectionary sugar and lecithin were slowly added to the humectant-syrup pre-mix and heated with agitation until the solids content reached about 80% by weight of the humectant-syrup pre-mix, resulting in a cooked humectant-syrup mixture. Shortening was added to the cooked humectant-syrup mixture and mixed until the shortening melted and was incorporated into the cooked humectant-syrup mixture.

A psyllium mixture was separately made by mixing the psyllium, citric acid, coloring agent, and flavoring agent. The psyllium mixture was then added to the cooked humectant-syrup mixture to create a final dough and mixed until homogenous. The resulting final dough was vigorously blended until a cohesive consistency was achieved. Then the final dough was spread in a tray and cut into pieces. The pieces were allowed to cool before individually wrapped in aluminum foil.

Texture Profile Analysis Method

In the Texture Profile Analysis Method, a mechanical compression tester is used to twice compress a specimen, and the resulting force is measured dynamically. The dynamic force data are then used to determine several parameters describing the texture profile of the sample.

The Texture Profile Analysis Method is conducted at 23° C. and 50% relative humidity. A tension/compression tester (such as TA-XT Plus Texture Analyzer, Stable Micro Systems, Godalming, Surrey, UK, or equivalent) outfitted with a 50-kgf tension/compression load cell is used in this method. The tension/compression tester is outfitted with a 19-mm (0.75-inch) diameter stainless-steel ball probe (such as TA-18A, Stable Micro Systems, Godalming, Surrey, UK, or equivalent) that serves as the upper member in the tension/compression. The bottom member of the tension/compression is the solid, flat base of the instrument. Force measurements are collected at a frequency of at least 200 Hz throughout the entire tension/compression procedure.

Samples are measured as received, and a specimen appropriate for measurement is one individual piece of soft chewable composition. A specimen is removed from packaging or sample jar and immediately analyzed without first being equilibrated to the lab environment. The specimen is placed beneath the center of the raised ball probe. The initial distance between the ball probe is and the base of the instrument, defined as the initial gap height, is 25 mm. (A larger initial gap height is used if necessary to accommodate the specimen.) The ball probe is moved downward at a rate of 1.0 mm/s. Immediately upon having measured a force of at least 5 gf the “trigger force,” the “first gap height” (tip of ball probe to base plate) of the compression tester is recorded, and the rate of the probe is increased to 2.0 mm/s. The specimen is compressed at this rate until 60% strain is reached. (Throughout this method, strain in a particular direction is defined as (d₀−dₛ)/d₀, expressed as a percent, where dₛ is the dimension of the gummy at the determination of strain and do is the corresponding initial dimension of the gummy before any deformation of the gummy was performed.) This is referred to as the “first compression.” Immediately, the probe direction is then reversed, and the probe is moved upward at 2.0 mm/s until the initial gap height is again achieved. During this upward stroke, a negative force may be recorded, associated with adhesion of the specimen to the probe. This movement is referred to as the “first upstroke.” The probe direction is then immediately reversed and is moved downward again, moving downward at 2.0 mm/s, and recording the “second gap height” (distance between the tip of ball probe and base plate) of the compression tester upon first measuring a force of greater than 5 gf. The specimen is compressed at this rate until 60% strain is achieved. This is referred to as the “second compression.” The probe is immediately reversed in direction, moving upward at 2.0 mm/s until the initial gap height is reached at which point the measurement is complete.

Immediately following the measurement portion the Texture Profile Analysis Method, the specimen is sealed in a container with minimal headspace. The Aw of the specimen is then determined using the Water Activity Method, and the resulting Aw is defined to be the Aw of the specimen analyzed in the Texture Profile Analysis Method.

The result of the measurement portion of the Texture Profile Analysis Method is a set of data in the form of recorded force versus time. These data are plotted with time on the horizontal axis and recorded force on the vertical axis. From these data, the following parameters are defined. The Hardness Parameter is defined as the maximum force measured during the first compression of the sample in gf reported to the nearest integer unit of gf. The Cohesiveness Parameter is defined as the dimensionless ratio, reported as a fractional value to two decimal places, of the work time area of the second compression to the work of the first compression, where the “work time area” of a compression is the integral of the measured force versus time from the trigger force until measured force falls to zero after 60% strain is achieved. The Springiness Parameter is the dimensionless ratio of the second gap height to the first gap height, reported as a fractional value to two decimal places. The Gumminess Parameter is the product of the Hardness Parameter and the Cohesiveness Parameter reported to the nearest integer unit of gf. The Adhesiveness Parameter is the work time area, reported to the nearest tenth of unit in gf s, associated with the first upstroke, where the work time area of an upstroke is the integral of the measured (generally negative) force versus time starting at the point at which measured force falls to zero after 60% strain is achieved until the completion of the upstroke.

Water Activity Test Method

The Aw of a specimen is defined as the ratio Aw=p/p₀, where p represents the partial pressure of water vapor in equilibrium with a specimen at a particular temperature and p₀ represents the partial pressure of water vapor pressure in equilibrium with pure water at that same temperature. The Aw level is therefore dimensionless; pure water has an Aw of unity, and a completely water-free substance has an Aw of zero. The water activity of a sample can thus be measured by measuring the relative humidity of the headspace when the sample reaches equilibrium, and the Aw is simply the RH expressed as a fractional value (between zero and unity). In this method, all samples are equilibrated to and all measurements performed at a temperature of 23° C. In this method, RH is measured using a RH probe containing a capacitive thin-film polymer sensor and an appropriate readout device (such as the Vaisala HMP42 probe and Vaisala HMI41 relative humidity indicator, Vaisala, Vantaa, Finland, or equivalents).

A sample removed from the packaging or vessel in which it is received and a specimen 75±25 g in mass is placed immediately in a 150-200 mL screw-top glass jar. The top is sealed quickly with parafilm. The RH probe is inserted through the parafilm and secured to prevent air transfer. After ten minutes of equilibration time, the RH reading is checked every two minutes. The first time the fractional RH reading is stable in the third decimal place for two consecutive readings, the RH is deemed to be stable, and the RH value is recorded. This fractional RH value is the Aw of the sample and is reported to two decimal places.

Swell Volume Method

The swell volume is measured as follows. A sample is grated into small pieces and 2.94 g is transferred to a 100 ml graduated mixing cylinder. Purified water is added to a total volume of 100 ml. The cylinder is capped, inverted ten times to obtain a uniform suspension and is allowed to stand at room temperature. At four and eight hours from the start of the test, the cylinder is inverted ten times again. After the eight hour inversion, the cylinder is allowed to stand at room temperature for 16 hours. The swell volume is read 24 hours after the start of the test and reported in whole milliliters. Any of the swelled mass that rose to the surface is added to the total swelled mass. 0.5 g of psyllium powder is tested in parallel for comparison. The samples are tested in triplicate and average values are reported.

Water Absorption Index Method

The water absorption index is measured as follows. Pre-weighed 50 ml conical tubes are filled with 35 ml of 25° C. purified water. A sample is grated into small pieces and 1 g is added to the conical tubes. The tubes are mixed by inversion five times. Then the tubes are placed in a water bath for 30 min. At 10, 20 and 30 min of incubation the solution in each tube is mixed times using a square spatula by stirring and lifting the contents from the bottom up to re-suspend any un-hydrated portion of the sample. Afterwards, the tubes are centrifuged at 700×g for 15 min. The water in each tube is decanted and the remaining gel is weighed. Psyllium powder is tested in parallel as a control. All samples are tested in triplicate and average values are reported. Water Absorption Index (WAI) is calculated by taking the quotient of the weight of the gel by the weight of the sample and normalized per one gram of psyllium.

Normalized WAI=(weight of gel/weight of sample)/(weight of psyllium per dose/weight of dose)

Particle Size Method

The particle size distribution of psyllium is determined by sieving. In this method, an air-jet sieve connected to vacuum-generating equipment is used to sequentially sieve a sample of psyllium, thereby establishing a distribution of psyllium particle size based on the mass of material lost in each sieving step.

An air-jet sieve (Hosokawa Micron Air-jet Sieve, Hosokawa Micron Powder Systems, Summit, NJ, or equivalent) is interfaced with a vacuum source (Pullman-Holt HEPA Vacuum Model 86, Pullman Ermator Inc., Tampa, FL, or equivalent). The air-jet sieve apparatus consists of a cylindrical base cavity onto which a 200-mm diameter sieve is placed. During the air-jet sieving process, the chamber defined by the base cavity volume and sieve volume is closed with an air-tight lid placed on top of the sieve. A vacuum of 7.0±0.4 inches (17.8±1.0 cm) of water below ambient pressure is maintained in the chamber from an opening in the base cavity, and a rotating wand containing an upward-facing slot and mounted in the center of the base cavity is rotated at 24 RPM. Through a hollow rotation shaft, the interior of the wand is connected directly to the ambient lab environment (pressure), and the air emerging from the slot in the wand both creates a localized fluidized bed in the particulate matter on the sieve screen directly above the slot and is the source of air pulled through the sieve elsewhere. The upward-facing slot in the wand is approximately 1.85 mm×100 mm in dimension, and the axis of rotation of the slot passes through one end of the slot such that in one complete rotation, the slot passes under the entire sieve screen area. The upward-facing slot is positioned at a distance of 5 to 6 mm beneath the underside of the sieve screen.

The set of sieves used in this analysis are U.S. Standard Sieves 200 mesh (75 μm), 140 mesh (106 μm), 100 mesh (150 μm), 80 mesh (180 μm), 70 mesh (212 μm), 60 mesh (250 μm), mesh (425 μm), 30 mesh (600 μm), 25 mesh (710 μm), 20 mesh (850 μm), and 18 mesh (1000 μm), and the sieves are used in this method in the order in which they appear in this listing.

The apparatus is outfitted with the initial 200 mesh (75 μm) sieve. A sample of psyllium with of mass 10.0±0.2 g and recorded to the nearest 0.01 g (defined as the “initial sample mass”) is introduced and spread across the sieve screen, the sieve is covered with the lid, and the air-jet sieving process is performed for 120 seconds. The mass of the psyllium retained on the mesh (the “remaining sample”) is then determined, the next coarser sieve is placed in the air-jet sieve apparatus, the remaining sample is then introduced and spread across the sieve screen, and the air-jet sieving process is performed for 120 seconds. This process continues, each time recording the incremental mass of material lost with the sieve used as well as the mass retained on the final sieve used.

The psyllium particle size distribution is determined as follows. The mass of material lost upon the first sieving (with 200 mesh sieve) is deemed to have a particle size less than 75 μm. The mass of material lost at subsequent sieving steps is deemed to have particle size smaller than the characteristic size of the sieve used in that sieving step but larger than the characteristic size of the sieve used in the previous step. (For example, the mass of material lost during sieving with the 100 mesh sieve is deemed to represent the fraction of material smaller than 150 μm but larger than 106 μm, which is the characteristic size of the 140 mesh sieve used previously in the sieving sequence.) Finally, the mass of material retained on the 18 mesh sieve at the end of the sieving procedure is deemed to have particle size greater than 1000 μm. Each of these sequential masses is divided by the initial sample mass of psyllium, yielding a dimensionless fractional value. Each fractional value then is multiplied by 100% and is reported as a percent, rounded to the nearest tenth of a percent.

### EXAMPLES

The following examples further describes and demonstrates an embodiment within the scope of the present invention. The example is given solely for the purpose of illustration and is not to be construed as a limitation of the present invention, as many variations thereof are possible without departing from the spirit and scope of the invention. All exemplified amounts are concentrations by weight of the total composition, i.e., wt/wt percentages, unless otherwise specified.

The following composition can be prepared in accordance with the present invention:

| | Ex. A |
|---|---|
| | (wt %) |
| Water | 10 |
| Corn Syrup 42 | 33 |
| DE | |
| Sucrose | 27 |
| Crisco ® | 5 |
| Shortening¹ | |
| Knox ® Gelatin | 3 |
| Citric Acid | 3 |
| Psyllium | 17.8 |
| Flavoring Agent | 0.11 |
| Coloring Agent | 0.11 |
| Potassium | 1 |
| Chloride | |
| Total | 100.00 |

¹Crisco ® Baking Sticks, Lot # 531342004 08:19 C.

Example A can be made according to the method of Examples 1-13. Salt can be added to the psyllium mixture before the psyllium mixture is added to the base syrup mixture.

| | Ex. B | Ex. C | Ex. D | Ex. E | Ex. F |
|---|---|---|---|---|---|
| | (wt %) | (wt %) | (wt %) | (wt %) | (wt %) |
| Water | 7.4 | 6.3 | 4.8 | 0 | 11 |
| Corn Syrup 42 DE | 24 | 19 | 7.5 | 15.58 | 25 |
| Sucrose | 8 | 5.1 | 0 | 0 | 8.68 |
| Crisco ® | 6 | 6 | 4 | 1 | 2 |
| Shortening¹ | | | | | |
| Knox ® Gelatin | 0 | 0 | 0 | 3 | 3 |
| Metamucil ® | 39.5 | 39.5 | 63.2 | 63.2 | 39.5 |
| Smooth Texture | | | | | |
| Sugar Orange⁴ | | | | | |
| Unagglomerated | 10.6 | 10.6 | 17 | 17 | 10.6 |
| Psyllium | | | | | |
| Flavoring Agent | 1 | 1 | 1 | 0.11 | 0.11 |
| Coloring Agent | 0.5 | 0.5 | 0.5 | 0.11 | 0.11 |
| Calcium Carbonate | 0 | 10 | 0 | 0 | 0 |
| Glycerin | 3 | 2 | 2 | 0 | 0 |
| Total | 100 | 100 | 100 | 100 | 100 |

¹Crisco ® Baking Sticks, Lot # 531342004 08:19 C.
⁴Distributed by the Procter and Gamble Co.

Examples B, C and D can be made according to the method of Examples 18-21. The agglomerated psyllium (Metamucil® Smooth Texture Sugar Orange) is added to the psyllium mixture along with the unagglomerated psyllium before it is added to the cooked humectant-syrup mixture. Calcium carbonate is added to the psyllium mixture before it is added to the cooked humectant-syrup mixture.

Examples E and F can be made according to Examples 1-13. The agglomerated psyllium (Metamucil® Smooth Texture Sugar Orange) is added to the psyllium mixture along with the unagglomerated psyllium before it is added to the base syrup mixture.

| | Ex. G | Ex. H |
|---|---|---|
| | (wt %) | (wt %) |
| Crystalline | 0 | 28 |
| Fructose | | |
| Water | 9.56 | 7 |
| Glycerin | 5 | 3 |
| Crisco ® | 7.7 | 5 |
| Shortening¹ | | |
| Monoglycerides | 0.46 | 3.5 |
| (Dimodan ® | | |
| SO/DK-A) | | |
| Lecithin (Solec ® | 0.92 | 0.5 |
| 152) | | |
| Isomalt | 0 | 18 |
| Sucralose | 0.05 | 0.05 |
| Erythritol | 13.4 | 0 |
| Calcium carbonate | 17.51 | 0 |
| Psyllium | 17 | 27 |
| Flavoring Agent | 1.9 | 1.9 |
| Citric Acid | 2.5 | 2 |
| Total | 100 | 100 |

¹Crisco ® Baking Sticks, Lot # 531342004 08:19 C.

Examples E and F can be made as follows. First, a processing aid pre-mix is made by blending the shortening and soy lecithin. The processing aid pre-mix is heated to about 45° C. or until the processing aid pre-mix is melted using a hot plate while continuously stirring. Then the psyllium is added to the processing aid pre-mix, resulting in a psyllium-processing aid mixture.

A humectant-syrup pre-mix is separately made by mixing glycerin, fructose, and water. The humectant-syrup pre-mix is heated while continuously stirring to about 65-75° C. until the solids content reached about 75-85% by weight of the humectant-syrup pre-mix, resulting in a cooked humectant-syrup mixture, or by formulating with the right amount of water to achieve the desired final solids content. The cooked humectant-syrup mixture is then added to the psyllium-processing aid mixture and mixed until homogenous. Then, the flavors, sweetener intensifiers such as sugar alcohols and the other remaining ingredients are added and mixed to form a final dough. The resulting final dough is vigorously blended until a cohesive consistency is achieved. Then the final dough is spread in a tray and cut into pieces or extruded, allowed to cool, and then individually wrapped in aluminum foil.

| | Ex. I | Ex. J | Ex. K | Ex. L | Ex. M |
|---|---|---|---|---|---|
| | (wt %) | (wt %) | (wt %) | (wt %) | (wt %) |
| Psyllium | 43.6 | 43.6 | 43.6 | 43.06 | 43.6 |
| Calcium Carbonate | 8.05 | 4.45 | 0 | 4 | 0 |
| Tricalcium | 0 | 0 | 0 | 4 | 8 |
| Phosphate | | | | | |
| Flavoring Agent | 0.04 | 0.04 | 0.04 | 0.04 | 0.04 |
| Citric Acid | 2.5 | 2.5 | 2.5 | 2.5 | 2.5 |
| Sucrose | 10 | 10 | 16.6 | 11 | 11 |
| Coloring Agent | 0 | 0.1 | 0.05 | 0.05 | 0.05 |
| Krystar ® Liquid | 26.6 | 28.3 | 28 | 26.14 | 25.6 |
| Fructose⁵ | | | | | |
| Glycerin | 2.18 | 3.98 | 2.18 | 2.18 | 2.18 |
| Soy Lecithin | 0.73 | 0.73 | 0.73 | 0.73 | 0.73 |
| Hydrogenated | 6 | 6 | 6 | 6 | 6 |
| Coconut Oil | | | | | |
| Mono- & di- | 0.3 | 0.3 | 0.3 | 0.3 | 0.3 |
| glycerides | | | | | |
| Total | 100 | 100 | 100 | 100 | 100 |

⁵Available from Tate & Lyle, London, UK

Examples I-M can be made as follows. First, a processing aid pre-mix is made by blending the hydrogenated coconut oil, mono- and di-glycerides, and soy lecithin. The processing aid pre-mix is heated to about 49° C. or until the processing aid pre-mix is melted using a hot plate while continuously stirring. Then the psyllium is added to the processing aid pre-mix resulting in a psyllium-processing aid mixture.

A humectant-syrup pre-mix is separately made by mixing glycerin, fructose and isomalt and water. The humectant-syrup pre-mix is heated while continuously stirring to about 65-75° C. until the solids content reached about 75-85% by weight of the humectant-syrup pre-mix, resulting in a cooked humectant-syrup mixture. The cooked humectant-syrup mixture is then added to the psyllium-processing aid mixture and mixed until homogenous. Then, the flavors, colors, and the remaining ingredients are added and mixed to form a final dough and the desired solids content is achieved. The resulting final dough is vigorously blended until a cohesive consistency is achieved. Then the final dough is extruded and formed into the desired shape, allowed to cool, and individually wrapped in aluminum foil.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about mm.”

Values disclosed herein as ends of ranges are not to be understood as being strictly limited to the exact numerical values recited. Instead, unless otherwise specified, each numerical range is intended to mean both the recited values and any real numbers including integers within the range. For example, a range disclosed as “1 to 10” is intended to mean “1, 2, 3, 4, 5, 6, 7, 8, 9, and 10” and a range disclosed as “1 to 2” is intended to mean “1.1, 1.2, 1.3, 1.4, 1.5, 1.6, 1.7, 1.8, 1.9, and 2.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A soft chewable composition comprising:
from about 1% to about 55% psyllium, by weight of the composition;
wherein the soft chewable composition has a Hardness Parameter of greater than about 300 gf at a water activity of about 0.80 and less than about 10,000 gf at a water activity of about 0.50 as measured by the Texture Profile Analysis Method.

2. The soft chewable composition of claim 1 further comprising about 0.10% to about 15% of a processing aid, by weight of the composition.

3. The soft chewable composition of claim 2 further comprising about 0.50% to about 8% of a processing aid, by weight of the composition.

4. The soft chewable composition of claim 3 further comprising less than about 20% of a binding agent, by weight of the composition; wherein the binding agent is selected from the group consisting of pectin, starch, gelatin, and combinations thereof.

5. The soft chewable composition of claim 1 comprising from about 1% to about 35% psyllium, by weight of the composition.

6. The soft chewable composition of claim 5 wherein the soft chewable composition has a Springiness Parameter of greater than about 0.40 at a water activity of about 0.80 and less than about 0.80 at a water activity of about 0.50 as measured by the Texture Profile Analysis Method.

7. The soft chewable composition of claim 1 comprising from about 1 g to about 45 g psyllium.

8. The soft chewable composition of claim 1 wherein the psyllium is substantially free of particles greater than about 250 μm.

9. The soft chewable composition of claim 1 further comprising liquid fructose.

10. A method of providing a health benefit to a user, comprising orally administering from about 1 to about 6 pieces of the soft chewable composition of claim 1, per day, to the user.

11. The method of claim 10, wherein the health benefit is selected from the group consisting providing digestive wellness, providing fiber, laxation, controlling blood glucose, increasing satiety, and any combination of the foregoing.

12. The method of claim 10 wherein the soft chewable composition comprises from about 1 g to about 11 g psyllium.

13. The method of claim 12 wherein the soft chewable composition is administered about 1 to about 3 times per day.

14. The method of claim 10 wherein the psyllium is substantially free of particles greater than about 250 μm.

15. A method of making a soft chewable composition comprising:
a. preparing a syrup pre-mixture comprising a humectant component and a carbohydrate;
b. heating the syrup pre-mixture to form a cooked syrup pre-mixture;
c. adding a processing aid to the cooked syrup pre-mixture and mixing until the processing aid is melted;
d. adding psyllium to the cooked syrup pre-mixture and mixing to form a final mixture;
e. optionally heating the final mixture to a temperature required to obtain a desired solids content;
f. forming the final mixture into a soft chewable composition; and
g. optionally post-processing the soft chewable composition.

16. The method of claim 15 further adding a pre-treated binding agent to the syrup pre-mixture before the heating step.

17. The method of claim 16 comprising from about 1% to about 55% psyllium, by weight of the composition.

18. The method of claim 17 wherein the psyllium comprises a particle distribution comprising greater than about 60% of particles within the range of about 75 μm to about 250 μm.

19. The method of claim 15 wherein the processing aid is hydrogenated palm oil.

20. The method of claim 15 wherein the processing aid and the psyllium are separately mixed in a mixing vessel before adding to the base syrup mixture.
